(19) United States
(12) Patent Application Publication
(10) Pub. No.: US 2010/0130482 A1
(43) Pub. Date: May 27, 2010

(54) DIAZABICYCLIC ARYL DERIVATIVES AS NICOTINIC ACETYLCHOLINE RECEPTOR LIGANDS

(76) Inventors: Dan Peters, Ballerup (DK); Gunnar M. Olsen, Ballerup (DK); Elsebet Østergaard Nielsen, Ballerup (DK); Tino Dyhring Jørgensen, Ballerup (DK); Philip K. Ahring, Ballerup (DK); Daniel B. Timmerman, Ballerup (DK)

Correspondence Address: BIRCH STEWART KOLASCH & BIRCH, PO BOX 747, FALLS CHURCH, VA, 22040-0747, US

(21) Appl. No.: 12/652,512

(22) Filed: Jan. 5, 2010

Related U.S. Application Data
(62) Division of application No. 10/586,749, filed on Jul. 21, 2006.
(60) Provisional application No. 60/541,754, filed on Feb. 5, 2004.
(60) Provisional application No. 60/574,946, filed on May 28, 2004.

(30) Foreign Application Priority Data
Feb. 4, 2004 (DK) PA 2004 00169
May 28, 2004 (DK) PA 2004 00839

(51) Int. Cl. A61K 31/5513; C07D 243/10; A61P 25/28; A61P 25/30; A61P 25/18; A61P 25/24

(52) U.S. Cl. 514/221; 540/556

(57) ABSTRACT

This invention relates to novel diazabicyclic aryl derivatives which are found to be cholinergic ligands at the nicotinic acetylcholine receptors. Due to their pharmacological profile the compounds of the invention may be useful for the treatment of diseases or disorders as diverse as those related to the cholinergic system of the central nervous system (CNS), the peripheral nervous system (PNS), diseases or disorders related to smooth muscle contraction, endocrine diseases or disorders, diseases or disorders related to neuro-degeneration, diseases or disorders related to inflammation, pain, and withdrawal symptoms caused by the termination of abuse of chemical substances.

### TECHNICAL FIELD

This invention relates to novel diazabicyclic aryl derivatives, which are found to be cholinergic ligands at the nicotinic acetylcholine receptors and modulators of the monoamine receptors and transporters. Due to their pharmacological profile the compounds of the invention may be useful for the treatment of diseases or disorders as diverse as those related to the cholinergic system of the central nervous system (CNS), the peripheral nervous system (PNS), diseases or disorders related to smooth muscle contraction, endocrine diseases or disorders, diseases or disorders related to neuro-degeneration, diseases or disorders related to inflammation, pain, and withdrawal symptoms caused by the termination of abuse of chemical substances.

### BACKGROUND ART

The endogenous cholinergic neurotransmitter, acetylcholine, exert its biological effect via two types of cholinergic receptors, the muscarinic Acetyl Choline Receptors (mAChR) and the nicotinic Acetyl Choline Receptors (nAChR).

As it is well established that muscarinic acetylcholine receptors dominate quantitatively over nicotinic acetylcholine receptors in the brain area important to memory and cognition, and much research aimed at the development of agents for the treatment of memory related disorders have focused on the synthesis of muscarinic acetylcholine receptor modulators.

Recently, however, an interest in the development of nAChR modulators has emerged. Several diseases are associated with degeneration of the cholinergic system i.e. senile dementia of the Alzheimer type, vascular dementia and cognitive impairment due to the organic brain damage disease related directly to alcoholism.

WO 00/58311 discloses 1,4-diazabicyclo[3.2.2]nonane-4-carboxylates and carboxamide derivatives useful as inhibitors of the nicotinic α7 receptor subtype. Other 1,4-diazabicyclo[3.2.2]nonane-4-methanone derivatives are not disclosed.

### SUMMARY OF THE INVENTION

The present invention is devoted to the provision novel modulators of the nicotinic and/or of the monoamine receptors, which modulators are useful for the treatment of diseases or disorders related to the cholinergic receptors, and in particular the nicotinic acetylcholine receptor (nAChR), the serotonin receptor (5-HTR), the dopamine receptor (DAR) and the norepinephrine receptor (NER), and of the biogenic amine transporters for serotonin (5-HT), dopamine (DA) and norepinephrine (NE).

Due to their pharmacological profile the compounds of the invention may be useful for the treatment of diseases or disorders as diverse as those related to the cholinergic system of the central nervous system (CNS), the peripheral nervous system (PNS), diseases or disorders related to smooth muscle contraction, endocrine diseases or disorders, diseases or disorders related to neuro-degeneration, diseases or disorders related to inflammation, pain, and withdrawal symptoms caused by the termination of abuse of chemical substances.

The compounds of the invention may also be useful as diagnostic tools or monitoring agents in various diagnostic methods, and in particular for in vivo receptor imaging (neuroimaging), and they may be used in labelled or unlabelled form.

In its first aspect the invention provides diazabicyclic aryl derivatives of Formula I

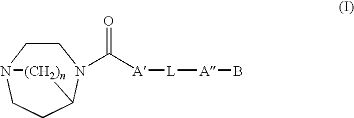

any of its enantiomers or any mixture of its enantiomers, an N-oxide, a prodrug, or a pharmaceutically-acceptable addition salt thereof, wherein

n is 1, 2 or 3;

A′ and A″, independently of one another, represent an aromatic monocyclic and/or polycyclic, carbocyclic and/or heterocyclic group, optionally substituted one or more times with substituents selected from the group consisting of alkyl, cycloalkyl, cycloalkyl-alkyl, hydroxy, alkoxy, hydroxyalkoxy, alkoxy-alkyl, alkoxy-alkoxy, cycloalkoxy, cycloalkoxy-alkyl, cycloalkoxy-alkoxy, halo, trihalomethyl, trihalomethoxy, cyano, nitro, amino, carboxy, amino-carbonyl (carbamoyl), sulfamoyl and phenyl; or with another monocyclic or polycyclic, carbocyclic or heterocyclic group; which additional monocyclic or polycyclic, carbocyclic or heterocyclic group may optionally be substituted one or more times with substituents selected from the group consisting of alkyl, cycloalkyl, cycloalkyl-alkyl, hydroxy, alkoxy, hydroxyalkoxy, alkoxy-alkyl, alkoxy-alkoxy, cycloalkoxy, cycloalkoxy-alkyl, cycloalkoxy-alkoxy, halo, trihalomethyl, trihalomethoxy, cyano, nitro, amino, carboxy, amino-carbonyl (carbamoyl), sulfamoyl and phenyl;

B represents a monocyclic heterocyclic group, optionally substituted one or more times with substituents selected from the group consisting of alkyl, cycloalkyl, cycloalkyl-alkyl, hydroxy, alkoxy, hydroxyalkoxy, alkoxy-alkyl, alkoxy-alkoxy, cycloalkoxy, cycloalkoxy-alkyl, cycloalkoxy-alkoxy, halo, trihalomethyl, trihalomethoxy, cyano, nitro, amino, oxo, carboxy, amino-carbonyl (carbamoyl), N-alkyl-amino-carbonyl (alkyl-carbamoyl), N,N-dialkyl-amino-carbonyl, alkyl-carbonyl-amino, alkyl-carbonyl-amino, sulfamoyl, phenyl or benzyl; or a group of formula —NR′—B′, —NR′—(C═V)—B′ or —NR′—(C═V)—NR″—B′; wherein R′ represents hydrogen, alkyl or a group of formula —(C═V)—NR″—B′; R″ represents hydrogen, alkyl, phenyl or benzyl; V represents O, S or NR′″; wherein R′″ represents hydrogen, alkyl or cyano; and B′ represents hydrogen, alkyl, alkenyl, alkynyl, cycloalkyl, cycloalkenyl, phenyl, benzyl or a monocyclic heterocyclic group; which phenyl, benzyl and heterocyclic groups are optionally substituted one or more times with substituents selected from the group consisting of alkyl, cycloalkyl, cycloalkyl-alkyl, hydroxy, alkoxy, hydroxyalkoxy, alkoxy-alkyl, alkoxy-alkoxy, cycloalkoxy, cycloalkoxy-alkyl, cycloalkoxy-alkoxy, halo, trihalomethyl, trihalomethoxy, cyano, nitro, amino, oxo, carboxy, amino-carbonyl (carbamoyl), N-alkyl-amino-carbonyl (alkyl-carbamoyl), N,N-dialkyl-amino-carbonyl, alkyl-carbonyl-amino, amino-carbonyl-amino (ureido), N-alkyl-amino-carbonyl-amino (N-alkyl-ureido), N,N-dialkyl-amino-carbonyl-amino (N,N-dialkyl-ureido), sulfamoyl, phenyl and benzyl; and

L represents a single (covalent) bond (i.e. L is absent); or a linking group selected from —CH₂—, —CH₂—CH₂—, —CH═CH—, —C≡C—, —Y—(CH₂)ₘ—, —(CH₂)ₘ—Y—, —CONR″″—, —NR″″CO—, —NR″″(SO₂)— and —(SO₂)NR″″—, wherein Y represents —O—, —S—, —SCH₂—, —SO—, —SO₂—, —NR″″—, R″″ represents hydrogen or alkyl; and m is 0, 1, 2 or 3.

In a second aspect the invention provides pharmaceutical compositions comprising a therapeutically effective amount of the diazabicyclic aryl derivative of the invention, or a pharmaceutically-acceptable addition salt thereof, together with at least one pharmaceutically-acceptable carrier or diluent.

Viewed from another aspect the invention relates to the use of the diazabicyclic aryl derivative of the invention, or a pharmaceutically-acceptable addition salt thereof, for the manufacture of pharmaceutical compositions/medicaments for the treatment, prevention or alleviation of a disease or a disorder or a condition of a mammal, including a human, which disease, disorder or condition is responsive to modulation of cholinergic receptors.

In yet another aspect the invention provides a method for treatment, prevention or alleviation of diseases, disorders or conditions of a living animal body, including a human, which disorder, disease or condition is responsive to modulation of cholinergic receptors, and which method comprises the step of administering to such a living animal body in need thereof a therapeutically effective amount of the diazabicyclic aryl derivative of the invention.

Other objects of the invention will be apparent to the person skilled in the art from the following detailed description and examples.

### DETAILED DISCLOSURE OF THE INVENTION

### Diazabicyclic Aryl Derivatives

In its first aspect the invention provides diazabicyclic aryl derivatives of Formula I

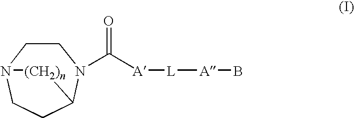

any of its enantiomers or any mixture of its enantiomers, an N-oxide, a prodrug, or a pharmaceutically-acceptable addition salt thereof, wherein

n is 1, 2 or 3;

A′ and A″, independently of one another, represent an aromatic monocyclic and/or polycyclic, carbocyclic and/or heterocyclic group, optionally substituted one or more times with substituents selected from the group consisting of alkyl, cycloalkyl, cycloalkyl-alkyl, hydroxy, alkoxy, hydroxyalkoxy, alkoxy-alkyl, alkoxy-alkoxy, cycloalkoxy, cycloalkoxy-alkyl, cycloalkoxy-alkoxy, halo, trihalomethyl, trihalomethoxy, cyano, nitro, amino, carboxy, amino-carbonyl (carbamoyl), sulfamoyl and phenyl; or with another monocyclic or polycyclic, carbocyclic or heterocyclic group; which additional monocyclic or polycyclic, carbocyclic or heterocyclic group may optionally be substituted one or more times with substituents selected from the group consisting of alkyl, cycloalkyl, cycloalkyl-alkyl, hydroxy, alkoxy, hydroxyalkoxy, alkoxy-alkyl, alkoxy-alkoxy, cycloalkoxy, cycloalkoxy-alkyl, cycloalkoxy-alkoxy, halo, trihalomethyl, trihalomethoxy, cyano, nitro, amino, carboxy, amino-carbonyl (carbamoyl), sulfamoyl and phenyl;

B represents a monocyclic heterocyclic group, optionally substituted one or more times with substituents selected from the group consisting of alkyl, cycloalkyl, cycloalkyl-alkyl, hydroxy, alkoxy, hydroxyalkoxy, alkoxy-alkyl, alkoxy-alkoxy, cycloalkoxy, cycloalkoxy-alkyl, cycloalkoxy-alkoxy, halo, trihalomethyl, trihalomethoxy, cyano, nitro, amino, oxo, carboxy, amino-carbonyl (carbamoyl), N-alkyl-amino-carbonyl (alkyl-carbamoyl), N,N-dialkyl-amino-carbonyl, alkyl-carbonyl-amino, alkyl-carbonyl-amino, sulfamoyl, phenyl or benzyl; or a group of formula —NR′—B′, —NR′—(C═V)—B′ or —NR′—(C═V)—NR″—B′; wherein R′ represents hydrogen, alkyl or a group of formula —(C═V)—NR″—B′; R″ represents hydrogen, alkyl, phenyl or benzyl; V represents O, S or NR′″; wherein R′″ represents hydrogen, alkyl or cyano; and B′ represents hydrogen, alkyl, alkenyl, alkynyl, cycloalkyl, cycloalkenyl, phenyl, benzyl or a monocyclic heterocyclic group; which phenyl, benzyl and heterocyclic groups are optionally substituted one or more times with substituents selected from the group consisting of alkyl, cycloalkyl, cycloalkyl-alkyl, hydroxy, alkoxy, hydroxyalkoxy, alkoxy-alkyl, alkoxy-alkoxy, cycloalkoxy, cycloalkoxy-alkyl, cycloalkoxy-alkoxy, halo, trihalomethyl, trihalomethoxy, cyano, nitro, amino, oxo, carboxy, amino-carbonyl (carbamoyl), N-alkyl-amino-carbonyl (alkyl-carbamoyl), N,N-dialkyl-amino-carbonyl, alkyl-carbonyl-amino, amino-carbonyl-amino (ureido), N-alkyl-amino-carbonyl-amino (N-alkyl-ureido), N,N-dialkyl-amino-carbonyl-amino (N,N-dialkyl-ureido), sulfamoyl, phenyl and benzyl; and

L represents a single (covalent) bond (i.e. L is absent); or a linking group selected from —CH₂—, —CH₂—CH₂—, —CH═CH—, —C≡C—, —Y—(CH₂)ₘ—, —(CH₂)ₘ—Y—, —CONR″″, —NR″″CO—, —NR″″(SO₂)— and —(SO₂)NR″″—, wherein Y represents —O—, —S—, —SCH₂—, —SO—, —SO₂—, —NR″″—; R″″ represents hydrogen or alkyl; and m is 0, 1, 2 or 3.

In one embodiment the diazabicyclic aryl derivative is a compound of Formula I, II, III or IV, as defined herein, wherein n is 1, 2 or 3.

In a more preferred embodiment n is 1 or 2.

In a most preferred embodiment n is 2.

In another embodiment the diazabicyclic aryl derivative is a compound of Formula I, II, III or IV, as defined herein, wherein L represents a single (covalent) bond (i.e. L is absent); or a linking group selected from —CH₂—, —CH₂—CH₂—, —CH═CH—, —C≡C—, —Y—(CH₂)ₘ—, —(CH₂)ₘ—Y—, —CONR″″—, —NR″″CO—, —NR″″(SO₂)— and —(SO₂)NR″″—, wherein Y represents —O—, —S—, —SCH₂—, —SO—, —SO₂—, —NR″″—; R″″ represents hydrogen or alkyl; and m is 0, 1, 2 or 3.

In a more preferred embodiment L represents a linking group selected from —CH═CH—, —C≡C—, —O—CH₂—, —CONH—, —NHCO—, —NH(SO₂)— and —(SO₂)NH—.

In another preferred embodiment L represents a single (covalent) bond (i.e. L is absent).

In a third embodiment the diazabicyclic aryl derivative is a compound of Formula I, II, III or IV, as defined herein, wherein A′ represents an aromatic monocyclic or polycyclic, carbocyclic or heterocyclic group, optionally substituted one or more times with substituents selected from the group consisting of alkyl, cycloalkyl, cycloalkyl-alkyl, hydroxy, alkoxy, hydroxyalkoxy, alkoxy-alkyl, alkoxy-alkoxy, cycloalkoxy, cycloalkoxy-alkyl, cycloalkoxy-alkoxy, halo, trihalomethyl, trihalomethoxy, cyano, nitro, amino, carboxy, amino-carbonyl (carbamoyl), sulfamoyl and phenyl; or with another monocyclic or polycyclic, carbocyclic or heterocyclic group; which additional monocyclic or polycyclic, carbocyclic or heterocyclic group may optionally be substituted one or more times with substituents selected from the group consisting of alkyl, cycloalkyl, cycloalkyl-alkyl, hydroxy, alkoxy, hydroxyalkoxy, alkoxy-alkyl, alkoxy-alkoxy, cycloalkoxy, cycloalkoxy-alkyl, cycloalkoxy-alkoxy, halo, trihalomethyl, trihalomethoxy, cyano, nitro, amino, carboxy, amino-carbonyl (carbamoyl), sulfamoyl and phenyl.

In a more preferred embodiment A′ represents an aromatic monocyclic carbocyclic group, optionally substituted one or more times with substituents selected from the group consisting of alkyl, cycloalkyl, cycloalkyl-alkyl, hydroxy, alkoxy, hydroxyalkoxy, alkoxy-alkyl, alkoxy-alkoxy, cycloalkoxy, cycloalkoxy-alkyl, cycloalkoxy-alkoxy, halo, trihalomethyl, trihalomethoxy, cyano, nitro, amino, carboxy, amino-carbonyl (carbamoyl), sulfamoyl and phenyl.

In an even more preferred embodiment A′ represents a phenyl or naphthyl group, optionally substituted one or more times with substituents selected from the group consisting of alkyl, cycloalkyl, cycloalkyl-alkyl, hydroxy, alkoxy, hydroxyalkoxy, alkoxy-alkyl, alkoxy-alkoxy, cycloalkoxy, cycloalkoxy-alkyl, cycloalkoxy-alkoxy, halo, trihalomethyl, trihalomethoxy, cyano, nitro, amino, carboxy, amino-carbonyl (carbamoyl), sulfamoyl and phenyl.

In a most preferred embodiment A′ represents a phen-1,4-diyl or naphth-2,6-diyl group.

In another preferred embodiment A′ represents an aromatic monocyclic heterocyclic group, optionally substituted one or more times with substituents selected from the group consisting of alkyl, cycloalkyl, cycloalkyl-alkyl, hydroxy, alkoxy, hydroxyalkoxy, alkoxy-alkyl, alkoxy-alkoxy, cycloalkoxy, cycloalkoxy-alkyl, cycloalkoxy-alkoxy, halo, trihalomethyl, trihalomethoxy, cyano, nitro, amino, carboxy, amino-carbonyl (carbamoyl), sulfamoyl and phenyl.

In a more preferred embodiment A′ represents a furanyl, pyrrolyl, isoxazolyl, 1,3,4-oxadiazolyl, 1,2,3-oxadiazolyl, pyridinyl, pyridinyl, pyridazinyl, indolyl, benzofuranyl, benzothienyl, quinoxalinyl or benzimidazolyl group.

In an even more preferred embodiment A′ represents a furan-2,5-diyl, furan-3,5-diyl, pyrrol-2,5-diyl, isoxazol-3,5-diyl, 1,3,4-oxadiazol-2,5-diyl, 1,2,3-oxadiazol-4,5-diyl, pyridin-2,5-diyl, pyridin-2,4-diyl, pyridazin-3,6-diyl, indol-2,5-diyl, benzofuran-2,5-diyl, benzothien-2,5-diyl, quinoxalin-2,6-diyl or benzimidazol-2,5-diyl group.

In a still more preferred embodiment A′ represents a group of formula

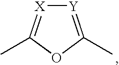

wherein X and Y, independently of one another, represent N and/or CR¹, wherein R¹ represents hydrogen, alkyl, cycloalkyl, cycloalkyl-alkyl, hydroxy, alkoxy, cycloalkoxy, cyanoalkyl, halo, haloalkyl, haloalkoxy, cyano, amino, nitro, aryl, aryloxy, heteroaryl or heteroaryloxy; which aryl, aryloxy, heteroaryl or heteroaryloxy may optionally be substituted one or two times with halo, haloalkyl, haloalkoxy, cyano, amino, nitro and/or a group of the formula —NCOR′″″, wherein R′″″ represents hydrogen or alkyl.

In another preferred embodiment A′ represents a group of formula

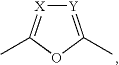

wherein X and Y, independently of one another, represent N and/or CR¹, wherein R¹ represents hydrogen, alkyl, cycloalkyl, cycloalkyl-alkyl, hydroxy, alkoxy, cycloalkoxy, cyanoalkyl, halo, haloalkyl, haloalkoxy, cyano, amino, nitro, phenyl or phenyloxy.

In a third preferred embodiment A′ represents a group of formula

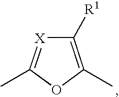

wherein X represents N or CR², wherein R² represents hydrogen, alkyl, cycloalkyl, cycloalkyl-alkyl, hydroxy, alkoxy, cycloalkoxy, cyanoalkyl, halo, haloalkyl, haloalkoxy, cyano, amino, nitro, aryl, aryloxy, heteroaryl or heteroaryloxy; which aryl, aryloxy, heteroaryl or heteroaryloxy may optionally be substituted one or two times with halo, haloalkyl, haloalkoxy, cyano, amino, nitro and/or a group of the formula —NCOR′″″, wherein R′″″ represents hydrogen or alkyl; and

In a more preferred embodiment R¹ represents hydrogen, alkyl, cycloalkyl, cycloalkyl-alkyl, hydroxy, alkoxy, cycloalkoxy, cyanoalkyl, halo, haloalkyl, haloalkoxy, cyano, amino, nitro, aryl, aryloxy, heteroaryl or heteroaryloxy; which aryl, aryloxy, heteroaryl or heteroaryloxy may optionally be substituted one or two times with halo, haloalkyl, haloalkoxy, cyano, amino, nitro and/or a group of the formula —NCOR′″″, wherein R′″″ represents hydrogen or alkyl.

In a fourth preferred embodiment A′ represents a group of formula

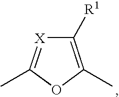

wherein X represents N or CR², wherein R² represents hydrogen, alkyl, cycloalkyl, cycloalkyl-alkyl, hydroxy, alkoxy, cycloalkoxy, cyanoalkyl, halo, haloalkyl, haloalkoxy, cyano, amino, nitro, phenyl or phenyloxy; and R¹ represents hydrogen, alkyl, cycloalkyl, cycloalkyl-alkyl, hydroxy, alkoxy, cycloalkoxy, cyanoalkyl, halo, haloalkyl, haloalkoxy, cyano, amino, nitro, phenyl or phenyloxy.

In a most preferred embodiment A′ represents furan-2,5-diyl.

In a fourth embodiment the diazabicyclic aryl derivative is a compound of Formula I, II, III or IV, as defined herein, wherein A″ represents an aromatic monocyclic or polycyclic, carbocyclic or heterocyclic group, optionally substituted one or more times with substituents selected from the group consisting of alkyl, cycloalkyl, cycloalkyl-alkyl, hydroxy, alkoxy, hydroxyalkoxy, alkoxy-alkyl, alkoxy-alkoxy, cycloalkoxy, cycloalkoxy-alkyl, cycloalkoxy-alkoxy, halo, trihalomethyl, trihalomethoxy, cyano, nitro, amino, carboxy, amino-carbonyl (carbamoyl), sulfamoyl and phenyl; or with another monocyclic or polycyclic, carbocyclic or heterocyclic group; which additional monocyclic or polycyclic, carbocyclic or heterocyclic group may optionally be substituted one or more times with substituents selected from the group consisting of alkyl, cycloalkyl, cycloalkyl-alkyl, hydroxy, alkoxy, hydroxyalkoxy, alkoxy-alkyl, alkoxy-alkoxy, cycloalkoxy, cycloalkoxy-alkyl, cycloalkoxy-alkoxy, halo, trihalomethyl, trihalomethoxy, cyano, nitro, amino, carboxy, amino-carbonyl (carbamoyl), sulfamoyl and phenyl.

In a more preferred embodiment A″ represents an aromatic monocyclic carbocyclic group, optionally substituted one or more times with substituents selected from the group consisting of alkyl, cycloalkyl, cycloalkyl-alkyl, hydroxy, alkoxy, hydroxyalkoxy, alkoxy-alkyl, alkoxy-alkoxy, cycloalkoxy, cycloalkoxy-alkyl, cycloalkoxy-alkoxy, halo, trihalomethyl, trihalomethoxy, cyano, nitro, amino, carboxy, amino-carbonyl (carbamoyl), sulfamoyl and phenyl.

In an even more preferred embodiment A″ represents a phenyl or naphthyl group; which aryl group is optionally substituted one or two times with substituents selected from the group consisting of alkyl, cycloalkyl, cycloalkyl-alkyl, hydroxy, alkoxy, hydroxyalkoxy, alkoxy-alkyl, alkoxy-alkoxy, cycloalkoxy, cycloalkoxy-alkyl, cycloalkoxy-alkoxy, halo, trihalomethyl, trihalomethoxy, cyano, nitro, amino, carboxy, amino-carbonyl (carbamoyl), sulfamoyl and phenyl.

In a most preferred embodiment A″ represents a phen-1,3-diyl or phen-1,4-diyl group.

In another preferred embodiment A″ represents a furanyl group; which group is optionally substituted one or two times with substituents selected from the group consisting of alkyl, cycloalkyl, cycloalkyl-alkyl, hydroxy, alkoxy, hydroxyalkoxy, alkoxy-alkyl, alkoxy-alkoxy, cycloalkoxy, cycloalkoxy-alkyl, cycloalkoxy-alkoxy, halo, trihalomethyl, trihalomethoxy, cyano, nitro, amino, carboxy, amino-carbonyl (carbamoyl), sulfamoyl and phenyl.

In a more preferred embodiment A″ represents a furan-2,5-diyl group; which group is optionally substituted one or two times with substituents selected from the group consisting of alkyl, cycloalkyl, cycloalkyl-alkyl, hydroxy, alkoxy, hydroxyalkoxy, alkoxy-alkyl, alkoxy-alkoxy, cycloalkoxy, cycloalkoxy-alkyl, cycloalkoxy-alkoxy, halo, trihalomethyl, trihalomethoxy, cyano, nitro, amino, carboxy, amino-carbonyl (carbamoyl), sulfamoyl and phenyl.

In a fifth embodiment the diazabicyclic aryl derivative is a compound of Formula I, wherein B represents a monocyclic heterocyclic group, optionally substituted one or more times with substituents selected from the group consisting of alkyl, cycloalkyl, cycloalkyl-alkyl, hydroxy, alkoxy, hydroxyalkoxy, alkoxy-alkyl, alkoxy-alkoxy, cycloalkoxy, cycloalkoxy-alkyl, cycloalkoxy-alkoxy, halo, trihalomethyl, trihalomethoxy, cyano, nitro, amino, oxo, carboxy, amino-carbonyl (carbamoyl), N-alkyl-amino-carbonyl (alkyl-carbamoyl), N,N-dialkyl-amino-carbonyl, alkyl-carbonyl-amino, sulfamoyl, phenyl and benzyl.

In a more preferred embodiment B represents a monocyclic heterocyclic group selected from pyrrolidinyl, pyrrolinyl, pyrrolyl, and pyridinyl; which monocyclic heterocyclic group is optionally substituted one or two times with substituents selected from the group consisting of alkyl, cycloalkyl, cycloalkyl-alkyl, hydroxy, alkoxy, hydroxyalkoxy, alkoxy-alkyl, alkoxy-alkoxy, cycloalkoxy, cycloalkoxy-alkyl, cycloalkoxy-alkoxy, halo, trihalomethyl, cyano, nitro, amino, oxo, carboxy, amino-carbonyl (carbamoyl), N-alkyl-amino-carbonyl (alkyl-carbamoyl), N,N-dialkyl-amino-carbonyl, alkyl-carbonyl-amino, sulfamoyl and phenyl.

In an even more preferred embodiment B represents a monocyclic heterocyclic group selected from pyrrolidinyl, pyrrolinyl and pyrrolyl; which monocyclic heterocyclic group is optionally substituted one or two times with substituents selected from the group consisting of alkyl, cycloalkyl, cycloalkyl-alkyl, hydroxy, alkoxy, hydroxyalkoxy, alkoxy-alkyl, alkoxy-alkoxy, cycloalkoxy, cycloalkoxy-alkyl, cycloalkoxy-alkoxy, halo, trihalomethyl, cyano, nitro, amino, oxo, carboxy, amino-carbonyl (carbamoyl), N-alkyl-amino-carbonyl (alkyl-carbamoyl), N,N-dialkyl-amino-carbonyl, alkyl-carbonyl-amino, sulfamoyl and phenyl.

In a still more preferred embodiment B represents a monocyclic heterocyclic group selected from pyrrolidinyl, 2-pyrrolinyl (4,5-dihydro-pyrrolyl), 3-pyrrolinyl (2,5-dihydro-pyrrolyl), pyrrolyl and pyridinyl; which monocyclic heterocyclic group is optionally substituted one or two times with substituents selected from the group consisting of alkyl, cycloalkyl, cycloalkyl-alkyl, hydroxy, alkoxy, hydroxyalkoxy, alkoxy-alkyl, alkoxy-alkoxy, cycloalkoxy, cycloalkoxy-alkyl, cycloalkoxy-alkoxy, halo, trihalomethyl, cyano, nitro, amino, oxo, carboxy, carbamoyl (amino-carbonyl), alkyl-carbamoyl (N-alkyl-amino-carbonyl), (N,N-dialkyl-amino-carbonyl), alkyl-carbonyl-amino, sulfamoyl and phenyl.

In a yet more preferred embodiment B represents 3-pyrrolinyl (2,5-dihydro-pyrrolyl) or pyridinyl (pyridin-4-yl); which monocyclic heterocyclic group is optionally substituted one or two times with substituents selected from the group consisting of alkyl, cycloalkyl, cycloalkyl-alkyl, hydroxy, alkoxy, hydroxyalkoxy, alkoxy-alkyl, alkoxy-alkoxy, cycloalkoxy, cycloalkoxy-alkyl, cycloalkoxy-alkoxy, halo, trihalomethyl, cyano, nitro, amino, oxo, carboxy, carbamoyl (amino-carbonyl), alkyl-carbamoyl (N-alkyl-amino-carbonyl), (N,N-dialkyl-amino-carbonyl), alkyl-carbonyl-amino, sulfamoyl and phenyl.

In a yet still more preferred embodiment B represents 3-pyrrolinyl (2,5-dihydro-pyrrolyl) or pyridinyl (pyridin-4-yl); which monocyclic heterocyclic group is optionally substituted one or two times with substituents selected from the group consisting of alkyl, hydroxy, alkoxy, halo, trihalomethyl, cyano, nitro, amino and/or oxo.

In a most preferred embodiment the diazabicyclic aryl derivative of the invention is
5-Hydroxy-1-{4-[5-(1-oxy-1,4-diaza-bicyclo[3.2.2]nonane-4-carbonyl)-furan-2-yl]-phenyl}-1,5-dihydro-pyrrol-2-one;
1-{4-[5-(1,4-Diaza-bicyclo[3.2.2]nonane-4-carbonyl)-furan-2-yl]-phenyl}-pyrrolidine-2,5-dione N-oxide; or
(1,4-Diaza-bicyclo[3.2.2]non-4-yl)-[5-(4-pyrrol-1-yl-phenyl)-furan-2-yl]-methanone;
or an enantiomer or a mixture of its enantiomers, or a pharmaceutically-acceptable addition salt thereof.

In a sixth embodiment the diazabicyclic aryl derivative is a compound of Formula I, wherein B represents a group of formula —NR′—B′, —NR′—(C═V)—B′ or —NR′—(C═V)—NR″—B′; wherein R′ represents hydrogen, alkyl or a group of formula —(C═V)—NR″—B′; R″ represents hydrogen, alkyl, phenyl or benzyl; V represents O, S or NR′″; wherein R′″ represents hydrogen, alkyl or cyano; and B′ represents hydrogen, alkyl, alkenyl, alkynyl, cycloalkyl, cycloalkenyl, phenyl, benzyl or a monocyclic heterocyclic group; which phenyl, benzyl and heterocyclic groups are optionally substituted one or more times with substituents selected from the group consisting of alkyl, cycloalkyl, cycloalkyl-alkyl, hydroxy, alkoxy, hydroxyalkoxy, alkoxy-alkyl, alkoxy-alkoxy, cycloalkoxy, cycloalkoxy-alkyl, cycloalkoxy-alkoxy, halo, trihalomethyl, trihalomethoxy, cyano, nitro, amino, oxo, carboxy, amino-carbonyl (carbamoyl), N-alkyl-amino-carbonyl (alkyl-carbamoyl), N,N-dialkyl-amino-carbonyl, alkyl-carbonyl-amino, amino-carbonyl-amino (ureido), N-alkyl-amino-carbonyl-amino (N-alkyl-ureido), N,N-dialkyl-amino-carbonyl-amino (N,N-dialkyl-ureido), sulfamoyl, phenyl and benzyl.

In a seventh embodiment the diazabicyclic aryl derivative is represented by Formula II

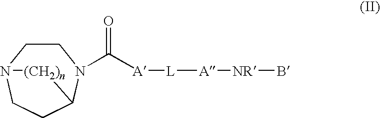

any of its enantiomers or any mixture of its enantiomers, or a prodrug, or a pharmaceutically-acceptable addition salt thereof, wherein n, A′, A″, L, R′ and B′ are as defined above.

In a more preferred embodiment L represents a single (covalent) bond (i.e. L is absent); R′ represents hydrogen or alkyl; and B′ represents hydrogen, alkyl, alkenyl, alkynyl, cycloalkyl, cycloalkenyl, phenyl, benzyl or a monocyclic heterocyclic group; which phenyl, benzyl and heterocyclic groups are optionally substituted one, two or three times with substituents selected from the group consisting of alkyl, cycloalkyl, cycloalkyl-alkyl, hydroxy, alkoxy, hydroxyalkoxy, alkoxy-alkyl, alkoxy-alkoxy, cycloalkoxy, cycloalkoxy-alkyl, cycloalkoxy-alkoxy, halo, trihalomethyl, trihalomethoxy, cyano, nitro, amino, oxo, carboxy, amino-carbonyl (carbamoyl), N-alkyl-amino-carbonyl (alkyl-carbamoyl), N,N-dialkyl-amino-carbonyl, alkyl-carbonyl-amino, sulfamoyl, phenyl and benzyl.

In an even more preferred embodiment B′ represents alkyl, phenyl, benzyl, furanyl, oxazolyl, isoxazolyl, thiazolyl, isothiazolyl, thiadiazolyl, imidazolyl, pyrazolyl, pyridinyl, pyrimidinyl or pyridazinyl; which phenyl, benzyl and heterocyclic groups are optionally substituted one or two times with substituents selected from the group consisting of alkyl, cycloalkyl, cycloalkyl-alkyl, hydroxy, alkoxy, hydroxyalkoxy, alkoxy-alkyl, alkoxy-alkoxy, cycloalkoxy, cycloalkoxy-alkyl, cycloalkoxy-alkoxy, halo, trihalomethyl, trihalomethoxy, cyano, nitro, amino, oxo, carboxy, amino-carbonyl (carbamoyl), N-alkyl-amino-carbonyl (alkyl-carbamoyl), N,N-dialkyl-amino-carbonyl, alkyl-carbonyl-amino, sulfamoyl, phenyl and benzyl.

In a still more preferred embodiment B′ represents furanyl, oxazolyl, isoxazolyl, thiazolyl, isothiazolyl, thiadiazolyl, imidazolyl, pyrazolyl, pyridinyl, pyrimidinyl or pyridazinyl; which heterocyclic group may optionally be substituted one or two times with alkyl, hydroxy-alkyl, cycloalkyl, cycloalkyl-alkyl, hydroxy, alkoxy, alkoxy-alkyl, cyanoalkyl, halo, trihalomethyl, trihalomethoxy, cyano, nitro, amino and/or phenyl.

In another preferred embodiment B′ represents alkyl, phenyl, benzyl or pyridinyl; which phenyl, benzyl and pyridinyl are optionally substituted with hydroxy, alkoxy, halo, trifluoromethyl, cyano, nitro, amino, N-alkyl-amino-carbonyl (alkyl-carbamoyl), N,N-dialkyl-amino-carbonyl, alkyl-carbonyl-amino, sulfamoyl, phenyl or benzyl.

In a third preferred embodiment B′ represents pyridin-2-yl, pyridin-3-yl, pyridin-4-yl; which pyridinyl may optionally be substituted one or two times with alkyl, hydroxy, alkoxy, halo, trihalomethyl, trihalomethoxy, nitro and/or amino.

In a fourth preferred embodiment n is 2; L represents a single (covalent) bond (i.e. L is absent); A′ represents a furanyl, oxazolyl or oxadiazolyl group; A″ represents a phenyl group; R′ represents hydrogen or alkyl; and B′ represents pyridin-2-yl, pyridin-3-yl, pyridin-4-yl; which pyridinyl may optionally be substituted one or two times with alkyl, hydroxy, alkoxy, halo, trihalomethyl, trihalomethoxy, nitro and/or amino.

In a most preferred embodiment the diazabicyclic aryl derivative of the invention is
(1,4-Diaza-bicyclo[3.2.2]non-4-yl)-{5-[4-(3-nitro-pyridin-2-ylamino)-phenyl]-furan-2-yl}-methanone;
or an enantiomer or a mixture of its enantiomers, or a pharmaceutically-acceptable addition salt thereof.

In an eighth embodiment the diazabicyclic aryl derivative is represented by Formula III

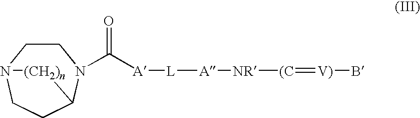

any of its enantiomers or any mixture of its enantiomers, or a prodrug, or a pharmaceutically-acceptable addition salt thereof, wherein n, A′, A″, L, R′, V and B′ are as defined above.

In a more preferred embodiment L represents a single (covalent) bond (i.e. L is absent); R′ represents hydrogen or alkyl; V represents O, S or NR′″; wherein R′″ represents hydrogen, alkyl or cyano; and B′ represents hydrogen, alkyl, alkenyl, alkynyl, cycloalkyl, cycloalkenyl, phenyl, benzyl or a monocyclic heterocyclic group; which phenyl, benzyl and heterocyclic groups are optionally substituted one, two or three times with substituents selected from the group consisting of alkyl, cycloalkyl, cycloalkyl-alkyl, hydroxy, alkoxy, hydroxyalkoxy, alkoxy-alkyl, alkoxy-alkoxy, cycloalkoxy, cycloalkoxy-alkyl, cycloalkoxy-alkoxy, halo, trihalomethyl, trihalomethoxy, cyano, nitro, amino, oxo, carboxy, amino-carbonyl (carbamoyl), N-alkyl-amino-carbonyl (alkyl-carbamoyl), N,N-dialkyl-amino-carbonyl, alkyl-carbonyl-amino, sulfamoyl, phenyl and benzyl.

In an even more preferred embodiment V represents O, S or NH; and B′ represents alkyl, alkenyl, cycloalkyl, cycloalkenyl, phenyl or benzyl; which phenyl and benzyl groups are optionally substituted one or two times with alkyl, hydroxy, alkoxy, alkoxy-alkyl, alkoxy-alkoxy, halo, trihalomethyl, trihalomethoxy, cyano, nitro, amino, amino-carbonyl (carbamoyl), N-alkyl-amino-carbonyl (alkyl-carbamoyl), N,N-dialkyl-amino-carbonyl and/or alkyl-carbonyl-amino; or furanyl, oxazolyl, isoxazolyl, thiazolyl, isothiazolyl, thiadiazolyl, imidazolyl, pyrazolyl, pyridinyl, pyrimidinyl or pyridazinyl; which heterocyclic group may optionally be substituted one or two times with alkyl, hydroxy-alkyl, cycloalkyl, cycloalkyl-alkyl, hydroxy, alkoxy, alkoxy-alkyl, cyanoalkyl, halo, trihalomethyl, trihalomethoxy, cyano, nitro, amino and/or phenyl.

In another preferred embodiment B′ represents a group of formula —CH₃, —CH₂CH₃, —CH═CH₂, —CH═CH—CH═CH₂, cyclopenta-1-enyl or cyclopenta-2,4-dienyl, phenyl or benzyl; which phenyl and benzyl may optionally be substituted one or two times with alkyl, hydroxy, alkoxy, halo, trihalomethyl, trihalomethoxy, nitro, amino, amino-carbonyl (amido), N-alkyl-amino-carbonyl (N-alkyl-amido), N,N-dialkyl-amino-carbonyl and/or alkyl-carbonyl-amino.

In a third preferred embodiment B′ represents phenyl, benzyl or pyridinyl; which phenyl, benzyl and pyridinyl groups are optionally substituted with halo, trifluoromethyl, cyano, nitro, amino, N-alkyl-amino-carbonyl (alkyl-carbamoyl), N,N-dialkyl-amino-carbonyl, alkyl-carbonyl-amino or sulfamoyl.

In a fourth preferred embodiment n is 2; L represents a single (covalent) bond (i.e. L is absent); A′ represents a furanyl, oxazolyl or oxadiazolyl group; A″ represents a phenyl group; R′ represents hydrogen or alkyl; V represents O, S or NH; and B′ represents a group of formula —CH₃, —CH₂CH₃, —CH═CH₂, —CH═CH—CH═CH₂, cyclopenta-1-enyl cyclopenta-2,4-dienyl, phenyl or benzyl; which phenyl and benzyl may optionally be substituted one or two times with alkyl, hydroxy, alkoxy, halo, trihalomethyl, trihalomethoxy, cyano, nitro, amino, amino-carbonyl (amido), N-alkyl-amino-carbonyl (N-alkyl-amido), N,N-dialkyl-amino-carbonyl (N,N-dialkyl-amido) and/or alkyl-carbonyl-amino.

In a most preferred embodiment the diazabicyclic aryl derivative of the invention is
N-{-4-[5-(1,4-Diaza-bicyclo[3.2.2]nonane-4-carbonyl)-furan-2-yl]-phenyl}-benzamide;
N-{3-[5-(1,4-Diaza-bicyclo[3.2.2]nonane-4-carbonyl)-furan-2-yl]-phenyl}-benzamide;
N-{4-[5-(1,4-Diaza-bicyclo[3.2.2]nonane-4-carbonyl)-furan-2-yl]-phenyl}-2-nitro-benzamide;
N-{4-[5-(1,4-Diaza-bicyclo[3.2.2]nonane-4-carbonyl)-furan-2-yl]-phenyl}-4-nitro-benzamide;
N-{4-[5-(1,4-Diaza-bicyclo[3.2.2]nonane-4-carbonyl)-furan-2-yl]-phenyl}-3-nitro-benzamide;
4-Amino-N-{4-[5-(1,4-diaza-bicyclo[3.2.2]nonane-4-carbonyl)-furan-2-yl]-phenyl}-benzamide;
3-Amino-N-{4-[5-(1,4-diaza-bicyclo[3.2.2]nonane-4-carbonyl)-furan-2-yl]-phenyl}-benzamide; or
N-{4-[5-(1,4-Diaza-bicyclo[3.2.2]nonane-4-carbonyl)-furan-2-yl]-phenyl}-isonicotinamide;
or an enantiomer or a mixture of its enantiomers, or a pharmaceutically-acceptable addition salt thereof.

In a ninth embodiment the diazabicyclic aryl derivative is represented by Formula IV

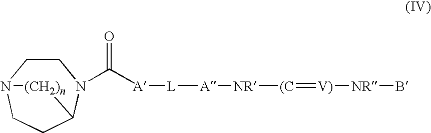

any of its enantiomers or any mixture of its enantiomers, or a prodrug, or a pharmaceutically-acceptable addition salt thereof, wherein n, A′, A″, L, R′, R″, V and B′ are as defined above.

In a more preferred embodiment L represents a single (covalent) bond (i.e. L is absent); R′ represents hydrogen, alkyl or a group of formula —(C═V)—NR″—B′; R″ represents hydrogen, alkyl, phenyl or benzyl; V represents O, S or NR′″; wherein R′″ represents hydrogen, alkyl or cyano; and B′ represents hydrogen, alkyl, alkenyl, alkynyl, cycloalkyl, cycloalkenyl, phenyl, benzyl or a monocyclic heterocyclic group; which phenyl, benzyl and heterocyclic groups are optionally substituted one, two or three times with substituents selected from the group consisting of alkyl, cycloalkyl, cycloalkyl-alkyl, hydroxy, alkoxy, hydroxyalkoxy, alkoxy-alkyl, alkoxy-alkoxy, cycloalkoxy, cycloalkoxy-alkyl, cycloalkoxy-alkoxy, halo, trihalomethyl, trihalomethoxy, cyano, nitro, amino, oxo, carboxy, amino-carbonyl (carbamoyl), N-alkyl-amino-carbonyl (alkyl-carbamoyl), N,N-dialkyl-amino-carbonyl, alkyl-carbonyl-amino, amino-carbonyl-amino (ureido), N-alkyl-amino-carbonyl-amino (N-alkyl-ureido), N,N-dialkyl-amino-carbonyl-amino (N,N-dialkyl-ureido), sulfamoyl, phenyl and benzyl.

In another preferred embodiment V represents O, S or NH; and B′ represents alkyl, alkenyl, cycloalkyl, cycloalkenyl, phenyl or benzyl; which phenyl and benzyl groups are optionally substituted one or two times with alkyl, hydroxy, alkoxy, alkoxy-alkyl, alkoxy-alkoxy, halo, trihalomethyl, trihalomethoxy, cyano, nitro, amino, amino-carbonyl (carbamoyl), N-alkyl-amino-carbonyl (alkyl-carbamoyl), N,N-dialkyl-amino-carbonyl, alkyl-carbonyl-amino, amino-carbonyl-amino (ureido), N-alkyl-amino-carbonyl-amino (N-alkyl-ureido) and/or N,N-dialkyl-amino-carbonyl-amino (N,N-dialkyl-ureido); or furanyl, oxazolyl, isoxazolyl, thiazolyl, isothiazolyl, thiadiazolyl, imidazolyl, pyrazolyl, pyridinyl, pyrimidinyl or pyridazinyl; which heterocyclic group may optionally be substituted one or two times with alkyl, hydroxy-alkyl, cycloalkyl, cycloalkyl-alkyl, hydroxy, alkoxy, alkoxy-alkyl, cyanoalkyl, halo, trihalomethyl, trihalomethoxy, cyano, nitro, amino and/or phenyl.

In a third preferred embodiment B′ represents a group of formula —CH₃, —CH₂CH₃, —CH═CH₂, —CH═CH—CH═CH₂, cyclopenta-1-enyl or cyclopenta-2,4-dienyl, phenyl or benzyl; which phenyl and benzyl may optionally be substituted one or two times with alkyl, hydroxy, alkoxy, halo, trihalomethyl, trihalomethoxy, cyano, nitro, amino, amino-carbonyl (amido), N-alkyl-amino-carbonyl (N-alkyl-amido), N,N-dialkyl-amino-carbonyl (N,N-dialkyl-amido) and/or alkyl-carbonyl-amino.

In a fourth preferred embodiment B′ represents alkyl, phenyl or benzyl; which phenyl and benzyl groups are optionally substituted one or two times with hydroxy, alkoxy, halo, trifluoromethyl, nitro, amino, alkyl-carbonyl-amino, amino-carbonyl-amino (ureido), N-alkyl-amino-carbonyl-amino (N-alkyl-ureido) and/or N,N-dialkyl-amino-carbonyl-amino (N,N-dialkyl-ureido).

In a fifth preferred embodiment n is 2; L represents a single (covalent) bond (i.e. L is absent); A′ represents a furanyl, oxazolyl, oxadiazolyl, thiazolyl or pyridazinyl group; A″ represents a phenyl group; and R′ represents hydrogen, alkyl or —(C═O)—NH—B′—; R″ represents hydrogen, alkyl, phenyl or benzyl; V represents O, S or NH; and B′ represents a group of formula —CH₃, —CH₂CH₃, —CH═CH₂, —CH═CH—CH═CH₂, cyclopenta-1-enyl cyclopenta-2,4-dienyl, phenyl or benzyl; which phenyl and benzyl may optionally be substituted one or two times with alkyl, hydroxy, alkoxy, halo, trihalomethyl, trihalomethoxy, cyano, nitro, amino, amino-carbonyl (amido), N-alkyl-amino-carbonyl (N-alkyl-amido), N,N-dialkyl-amino-carbonyl (N,N-dialkyl-amido) and/or alkyl-carbonyl-amino.

In a sixth preferred embodiment n is 2; L represents a single (covalent) bond (i.e. L is absent); A′ represents a furanyl, oxazolyl, oxadiazolyl, thiazolyl or pyridazinyl group; A″ represents a phenyl group; R′ represents hydrogen, alkyl or —(C═O)—NH—B′—; R″ represents hydrogen, alkyl, phenyl or benzyl; V represents O, S or NH; and B′ represents a group of formula —CH₃, —CH₂CH₃, —CH═CH₂, —CH═CH—CH═CH₂, cyclopenta-1-enyl cyclopenta-2,4-dienyl, phenyl or benzyl; which phenyl and benzyl may optionally be substituted one or two times with alkyl, hydroxy, alkoxy, halo, trihalomethyl, trihalomethoxy, cyano, nitro, amino, amino-carbonyl (amido), N-alkyl-amino-carbonyl (N-alkyl-amido), N,N-dialkyl-amino-carbonyl (N,N-dialkyl-amido).

In a seventh preferred embodiment n is 2; L represents a single (covalent) bond (i.e. L is absent); A′ represents a furanyl, oxazolyl, oxadiazolyl, thiazolyl or pyridazinyl group; A″ represents a phenyl group; and R′ represents hydrogen, alkyl or —(C═O)—NH—B′—; R″ represents hydrogen, alkyl, phenyl or benzyl; V represents O, S or NH; and B′ represents a group of formula —CH₃, —CH₂CH₃, —CH═CH₂, —CH═CH—CH═CH₂, cyclopenta-1-enyl cyclopenta-2,4-dienyl, phenyl or benzyl; which phenyl and benzyl may optionally be substituted one or two times with alkyl, hydroxy, alkoxy, halo, trihalomethyl, trihalomethoxy, cyano, nitro, amino, amino-carbonyl (amido), N-alkyl-amino-carbonyl (N-alkyl-amido), N,N-dialkyl-amino-carbonyl (N,N-dialkyl-amido) and/or alkyl-carbonyl-amino.

In an eighth preferred embodiment B′ represents alkyl, phenyl, benzyl or pyridyl; which phenyl, benzyl and pyridyl groups are optionally substituted one or two times with substituents selected from the group consisting of hydroxy, alkoxy, halo, trifluoromethyl, nitro, amino, alkyl-carbonyl-amino, N-alkyl-amino-carbonyl-amino (N-alkyl-ureido), N,N-dialkyl-amino-carbonyl-amino (N,N-dialkyl-ureido) and sulfamoyl.

In a most preferred embodiment the diazabicyclic aryl derivative of the invention is
1-{4-[5-(1,4-Diaza-bicyclo[3.2.2]nonane-4-carbonyl)-furan-2-yl]-phenyl}-3-ethyl-urea;
1-{4-[5-(1,4-Diaza-bicyclo[3.2.2]nonane-4-carbonyl)-furan-2-yl]-phenyl}-3-phenyl-urea;
1-{4-[5-(1,4-Diaza-bicyclo[3.2.2]nonane-4-carbonyl)-furan-2-yl]-phenyl}-3-(2-nitrophenyl)-urea;
1-{4-[5-(1,4-Diaza-bicyclo[3.2.2]nonane-4-carbonyl)-furan-2-yl]-phenyl}-3-(2-acetylaminophenyl)-urea;
1-{4-[5-(1,4-Diaza-bicyclo[3.2.2]nonane-4-carbonyl)-furan-2-yl]-phenyl}-3-(2-aminophenyl)-urea;
1-{4-[5-(1,4-Diaza-bicyclo[3.2.2]nonane-4-carbonyl)-furan-2-yl]-phenyl}-3-(5-chloro-2-methoxyphenyl)-thiourea;
1-{4-[5-(1,4-Diaza-bicyclo[3.2.2]nonane-4-carbonyl)-furan-2-yl]-phenyl}-3-(5-chloro-2-methoxy-phenyl)-urea;
1-{4-[5-(1,4-Diaza-bicyclo[3.2.2]nonane-4-carbonyl)-furan-2-yl]-phenyl}-3-benzyl-urea;
1-{4-[5-(1,4-Diaza-bicyclo[3.2.2]nonane-4-carbonyl)-furan-2-yl]-phenyl}-1′-benzylaminocarbonyl-3-benzyl-urea;
1-{4-[5-(1,4-Diaza-bicyclo[3.2.2]nonane-4-carbonyl)-furan-2-yl]-phenyl}-3-(2-chlorophenyl)-urea;
1-{3-[5-(1,4-Diaza-bicyclo[3.2.2]nonane-4-carbonyl)-furan-2-yl]-phenyl}-3-phenyl-urea;
1-{4-[5-(1,4-Diaza-bicyclo[3.2.2]nonane-4-carbonyl)-furan-2-yl]-phenyl}-3-(2-fluorophenyl)-urea;
1-{4-[5-(1,4-Diaza-bicyclo[3.2.2]nonane-4-carbonyl)-furan-2-yl]-phenyl}-3-(3-fluorophenyl)-urea;
1-{4-[5-(1,4-Diaza-bicyclo[3.2.2]nonane-4-carbonyl)-furan-2-yl]-phenyl}-3-(2-trifluoromethylphenyl)-urea;
1-[2-(3-{4-[5-(1,4-Diaza-bicyclo[3.2.2]nonane-4-carbonyl)-furan-2-yl]-phenyl}-ureido)-phenyl]-3-ethyl-urea;
1-{4-[5-(1,4-Diaza-bicyclo[3.2.2]nonane-4-carbonyl)-furan-2-yl]-phenyl}-3-(3-trifluoromethylphenyl)-urea; or
1-{3-[5-(1,4-Diaza-bicyclo[3.2.2]nonane-4-carbonyl)-furan-2-yl]-phenyl}-3-ethyl-urea;
or an enantiomer or a mixture of its enantiomers, or a pharmaceutically-acceptable addition salt thereof.

Any combination of two or more of the embodiments described herein is considered within the scope of the present invention.

### Definition of Substituents

In the context of this invention an alkyl group designates a univalent saturated, straight or branched hydrocarbon chain. The hydrocarbon chain preferably contain of from one to eighteen carbon atoms (C₁₋₁₈-alkyl), more preferred of from one to six carbon atoms (C₁₋₆-alkyl; lower alkyl), including pentyl, isopentyl, neopentyl, tertiary pentyl, hexyl and isohexyl. In a preferred embodiment alkyl represents a C₁₋₄-alkyl group, including butyl, isobutyl, secondary butyl, and tertiary butyl. In another preferred embodiment of this invention alkyl represents a C₁₋₃-alkyl group, which may in particular be methyl, ethyl, propyl or isopropyl.

In the context of this invention a cycloalkyl group designates a cyclic alkyl group, preferably containing of from three to seven carbon atoms (C₃₋₇-cycloalkyl), including cyclopropyl, cyclobutyl, cyclopentyl, cyclohexyl and cycloheptyl.

In the context of this invention a cycloalkyl-alkyl group designates a cycloalkyl group as defined above, which cycloalkyl group is substituted on an alkyl group as also defined above. Examples of preferred cycloalkyl-alkyl groups of the invention include cyclopropylmethyl and cyclopropylethyl.

In the context of this invention an alkoxy group designates an “alkyl-O—” group, wherein alkyl is as defined above. Examples of preferred alkoxy groups of the invention include methoxy and ethoxy.

In the context of this invention a cycloalkoxy group designates a “cycloalkyl-O—” group, wherein cycloalkyl is as defined above.

In the context of this invention a cyano-alkyl group designates an alkyl group substituted with CN, wherein alkyl is as defined above.

In the context of this invention halo represents fluoro, chloro, bromo or iodo, and haloalkyl group designates an alkyl group as defined herein, which alkyl group is substituted one or more times with halo. Thus a trihalomethyl group represents e.g. a trifluoromethyl group, a trichloromethyl group, and similar trihalo-substituted methyl groups. Preferred haloalkyl groups of the invention include trihalogenmethyl, preferably —CF₃.

In the context of this invention a haloalkoxy group designates an alkoxy group as defined herein, which alkoxy group is substituted one or more times with halo. Preferred haloalkoxy groups of the invention include trihalogenmethoxy, preferably —OCF₃.

In the context of this invention an aryl group designates a monocyclic or polycyclic aromatic hydrocarbon group. Examples of preferred aryl groups of the invention include phenyl, indenyl, naphthyl, azulenyl, fluorenyl, and anthracenyl. The most preferred aryl group of the invention is phenyl.

In the context of this invention an aryloxy group designates an “aryl-O—” group, wherein aryl is as defined above. The most preferred aryloxy group of the invention is phenoxy.

In the context of this invention a heteroaryl group designates an aromatic mono- or polycyclic heterocyclic group, which holds one or more heteroatoms in its ring structure. Preferred heteroatoms include nitrogen (N), oxygen (O) and sulphur (S).

Preferred 5-6 membered heteroaryl groups of the invention include furanyl, in particular furan-2- or 3-yl; thienyl, in particular thien-2- or 3-yl; selenophenyl, in particular selenophen-2- or 3-yl; pyrrolyl (azolyl), in particular pyrrol-2- or 3-yl; oxazolyl, in particular oxazol-2,4- or 5-yl; thiazolyl, in particular thiazol-2,4- or 5-yl; imidazolyl, in particular imidazol-2- or 4-yl; pyrazolyl, in particular pyrazol-3- or 4-yl; isoxazolyl, in particular isoxazol-3,4- or 5-yl; isothiazolyl, in particular isothiazol-3-, 4- or 5-yl; oxadiazolyl, in particular 1,2,3-oxadiazol-4- or 5-yl, or 1,3,4-oxadiazol-2-yl; triazolyl, in particular 1,2,3-triazol-4-yl or 1,2,4-triazol-3-yl; thiadiazolyl, in particular 1,2,3-thiadiazol-4- or 5-yl, or 1,3,4-thiadiazol-2-yl; pyridyl, in particular pyrid-2-, 3- or 4-yl; pyridazinyl, in particular pyridazin-3- or 4-yl; pyrimidinyl, in particular pyrimidin-2-, 4- or 5-yl; pyrazinyl, in particular pyrazin-2- or 3-yl; and triazinyl, in particular 1,2,4- or 1,3,5-triazinyl.

More preferred 5 membered heteroaryl groups of the invention include furanyl, in particular furan-2- or 3-yl; thienyl, in particular thien-2- or 3-yl; pyrrolyl (azolyl), in particular pyrrol-2- or 3-yl; oxazolyl, in particular oxazol-2,4- or 5-yl; thiazolyl, in particular thiazol-2,4- or 5-yl; isoxazolyl, in particular isoxazol-3,4- or 5-yl; isothiazolyl, in particular isothiazol-3-, 4- or 5-yl; and thiadiazolyl, in particular 1,2,3-thiadiazol-4- or 5-yl, or 1,3,4-thiadiazol-2-yl.

Yet more preferred 5 membered heteroaryl groups of the invention include furanyl, thienyl, pyrrolyl, oxazolyl and oxadiazolyl.

Most preferred 5 membered heteroaryl groups of the invention include furanyl, in particular furan-2- or 3-yl; and thienyl, in particular thien-2- or 3-yl.

More preferred 6 membered heteroaryl groups of the invention include pyridyl, in particular pyrid-2-, 3- or 4-yl; and pyrazinyl, in particular pyrazin-2- or 3-yl.

In the context of this invention an aromatic bicyclic heterocyclic group designates a bicyclic heterocyclic group, which holds one or more heteroatoms in its ring structure. In the context of this invention the term “bicyclic heterocyclic group” includes benzo-fused five- and six-membered heterocyclic rings containing one or more heteroatoms. Preferred heteroatoms include nitrogen (N), oxygen (O), and sulphur (S).

Preferred bicyclic heteroaryl groups of the invention include indolizinyl, in particular indolizin-2-, 5- or 6-yl; indolyl, in particular indol-2-, 5- or 6-yl; isoindolyl, in particular isoindol-2-, 5- or 6-yl; benzo[b]furanyl, in particular benzofuran-2-, 5- or 6-yl; benzo[b]thienyl, in particular benzothien-2-, 5- or 6-yl; benzoimidazolyl, in particular benzoimidazol-2-, 5- or 6-yl; benzothiazolyl, in particular benzothiazol-5- or 6-yl; purinyl, in particular purin-2- or 8-yl; quinolinyl, in particular quinolin-2-, 3-, 6- or 7-yl; isoquinolinyl, in particular isoquinolin-3-, 6- or 7-yl; cinnolinyl, in particular cinnolin-6- or 7-yl; phthalazinyl, in particular phthalazin-6- or 7-yl; quinazolinyl, in particular quinazolin-2-, 6- or 7-yl; quinoxalinyl, in particular quinoxalin-2- or 6-yl; 1,8-naphthyridinyl, in particular 1,8-naphthyridin-2-, 3-, 6- or 7-yl; and pteridinyl, in particular pteridin-2-, 6- or 7-yl.

More preferred bicyclic heteroaryl groups of the invention include indolyl, in particular indol-2-, 5- or 6-yl; benzo[b]furanyl, in particular benzofuran-2-, 5- or 6-yl; benzo[b]thienyl, in particular benzothien-2-, 5- or 6-yl; benzoimidazolyl, in particular benzoimidazol-2-, 5- or 6-yl; and quinoxalinyl, in particular quinoxalin-2- or 6-yl.

Most preferred bicyclic heteroaryl groups of the invention include indolyl, in particular indol-2-, 5- or 6-yl; benzo[b]furanyl, in particular benzofuran-2-, 5- or 6-yl; benzo[b]thienyl, in particular benzothien-2-, 5- or 6-yl.

In the context of this invention a heteroaryloxy group designates a “heteroaryl-O—” group, wherein heteroaryl is as defined above.

### Pharmaceutically Acceptable Salts

The diazabicyclic aryl derivative of the invention may be provided in any form suitable for the intended administration. Suitable forms include pharmaceutically (i.e. physiologically) acceptable salts, and pre- or prodrug forms of the chemical compound of the invention.

Examples of pharmaceutically acceptable addition salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate derived, the phthalate, the salicylate, the sorbate, the stearate, the succinate, the tartrate, the toluene-p-sulphonate, and the like. Such salts may be formed by procedures well known and described in the art.

Metal salts of a chemical compound of the invention include alkali metal salts, such as the sodium salt of a chemical compound of the invention containing a carboxy group.

In the context of this invention the “onium salts” of N-containing compounds may also be contemplated as pharmaceutically acceptable salts. Preferred “onium salts” include the alkyl-onium salts, the cycloalkyl-onium salts, and the cycloalkylalkyl-onium salts. Particularly preferred onium salts of the invention include those created at the N′ position according to the following Formula I′

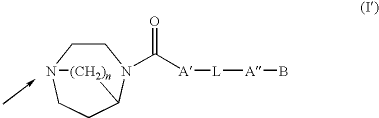

### Steric Isomers

The chemical compounds of the present invention may exist in (+) and (−) forms as well as in racemic forms. The racemates of these isomers and the individual isomers themselves are within the scope of the present invention.

Racemic forms can be resolved into the optical antipodes by known methods and techniques. One way of separating the diastereomeric salts is by use of an optically active acid, and liberating the optically active amine compound by treatment with a base. Another method for resolving racemates into the optical antipodes is based upon chromatography on an optical active matrix. Racemic compounds of the present invention can thus be resolved into their optical antipodes, e.g., by fractional crystallisation of d- or l- (tartrates, mandelates, or camphorsulphonate) salts for example.

The chemical compounds of the present invention may also be resolved by the formation of diastereomeric amides by reaction of the chemical compounds of the present invention with an optically active activated carboxylic acid such as that derived from (+) or (−) phenylalanine, (+) or (−) phenylglycine, (+) or (−) camphanic acid or by the formation of diastereomeric carbamates by reaction of the chemical compound of the present invention with an optically active chloroformate or the like.

Additional methods for the resolving the optical isomers are known in the art. Such methods include those described by Jaques J, Collet A, & Wilen S in “Enantiomers, Racemates, and Resolutions”, John Wiley and Sons, New York (1981).

Optical active compounds can also be prepared from optical active starting materials.

### Methods of Producing Diazabicyclic Aryl Derivatives

The diazabicyclic aryl derivative of the invention may be prepared by conventional methods for chemical synthesis, e.g. those described in the working examples. The starting materials for the processes described in the present application are known or may readily be prepared by conventional methods from commercially available chemicals.

Also one compound of the invention can be converted to another compound of the invention using conventional methods.

The end products of the reactions described herein may be isolated by conventional techniques, e.g. by extraction, crystallisation, distillation, chromatography, etc.

### Biological Activity

The present invention is devoted to the provision novel ligands and modulators of the nicotinic receptors, which ligands and modulators are useful for the treatment of diseases or disorders related to the cholinergic receptors, and in particular the nicotinic acetylcholine receptor (nAChR). Preferred compounds of the invention show a pronounced nicotinic acetylcholine α7 receptor subtype selectivity.

The compounds of the present invention may in particular be agonists, partial agonists, antagonists and/or allosteric modulators of the nicotinic acetylcholine receptor.

Due to their pharmacological profile the compounds of the invention may be useful for the treatment of diseases or disorders as diverse as those related to the cholinergic system of the central nervous system (CNS), the peripheral nervous system (PNS), diseases or disorders related to smooth muscle contraction, endocrine diseases or disorders, diseases or disorders related to neuro-degeneration, diseases or disorders related to inflammation, pain, and withdrawal symptoms caused by the termination of abuse of chemical substances.

The compounds of the invention may also be useful as diagnostic tools or monitoring agents in various diagnostic methods, and in particular for in vivo receptor imaging (neuroimaging), and they may be used in labelled or unlabelled form.

In a preferred embodiment the compounds of the invention are used for the treatment of diseases, disorders, or conditions relating to the central nervous system. Such diseases or disorders includes anxiety, cognitive disorders, learning deficit, memory deficits and dysfunction, Alzheimer's disease, attention deficit, attention deficit hyperactivity disorder (ADHD), Parkinson's disease, Huntington's disease, Amyotrophic Lateral Sclerosis, Gilles de la Tourette's syndrome, psychosis, depression, mania, manic depression, schizophrenia, obsessive compulsive disorders (OCD), panic disorders, eating disorders such as anorexia nervosa, bulimia and obesity, narcolepsy, nociception, AIDS-dementia, senile dementia, periferic neuropathy, autism, dyslexia, tardive dyskinesia, hyperkinesia, epilepsy, bulimia, post-traumatic syndrome, social phobia, sleeping disorders, pseudodementia, Ganser's syndrome, pre-menstrual syndrome, late luteal phase syndrome, chronic fatigue syndrome, mutism, trichotillomania, and jet-lag.

In a preferred embodiment diseases, disorders, or conditions relating to the central nervous system for which the compounds of the invention are used are cognitive disorders, psychosis, schizophrenia and/or depression.

In another preferred embodiment the compounds of the invention may be useful for the treatment of diseases, disorders, or conditions associated with smooth muscle contractions, including convulsive disorders, angina pectoris, premature labour, convulsions, diarrhoea, asthma, epilepsy, tardive dyskinesia, hyperkinesia, premature ejaculation, and erectile difficulty.

In yet another preferred embodiment the compounds of the invention may be useful for the treatment of endocrine disorders, such as thyrotoxicosis, pheochromocytoma, hypertension and arrhythmias.

In still another preferred embodiment the compounds of the invention may be useful for the treatment of neurodegenerative disorders, including transient anoxia and induced neuro-degeneration.

In even another preferred embodiment the compounds of the invention may be useful for the treatment of inflammatory diseases, disorders, or conditions, including inflammatory skin disorders such as acne and rosacea, Chron's disease, inflammatory bowel disease, ulcerative colitis, and diarrhoea.

In still another preferred embodiment the compounds of the invention may be useful for the treatment of mild, moderate or even severe pain of acute, chronic or recurrent character, as well as pain caused by migraine, postoperative pain, and phantom limb pain. The pain may in particular be neuropathic pain, chronic headache, central pain, pain related to diabetic neuropathy, to post therapeutic neuralgia, or to peripheral nerve injury.

Finally the compounds of the invention may be useful for the treatment of withdrawal symptoms caused by termination of use of addictive substances. Such addictive substances include nicotine containing products such as tobacco, opioids such as heroin, cocaine and morphine, benzodiazepines and benzodiazepine-like drugs, and alcohol. Withdrawal from addictive substances is in general a traumatic experience characterised by anxiety and frustration, anger, anxiety, difficulties in concentrating, restlessness, decreased heart rate and increased appetite and weight gain.

In this context “treatment” covers treatment, prevention, prophylactics and alleviation of withdrawal symptoms and abstinence as well as treatment resulting in a voluntary diminished intake of the addictive substance.

In another aspect, the compounds of the invention are used as diagnostic agents, e.g. for the identification and localisation of nicotinic receptors in various tissues.

### Pharmaceutical Compositions

In another aspect the invention provides novel pharmaceutical compositions comprising a therapeutically effective amount of diazabicyclic aryl derivative of the invention.

While a chemical compound of the invention for use in therapy may be administered in the form of the raw chemical compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the diazabicyclic aryl derivative of the invention, or a pharmaceutically acceptable salt or derivative thereof, together with one or more pharmaceutically acceptable carriers therefore, and, optionally, other therapeutic and/or prophylactic ingredients, know and used in the art. The carrier(s) must be “acceptable” in the sense of being compatible with the other ingredients of the formulation and not harmful to the recipient thereof.

The pharmaceutical composition of the invention may be administered by any convenient route, which suits the desired therapy. Preferred routes of administration include oral administration, in particular in tablet, in capsule, in dragé, in powder, or in liquid form, and parenteral administration, in particular cutaneous, subcutaneous, intramuscular, or intravenous injection. The pharmaceutical composition of the invention can be manufactured by the skilled person by use of standard methods and conventional techniques appropriate to the desired formulation. When desired, compositions adapted to give sustained release of the active ingredient may be employed.

Further details on techniques for formulation and administration may be found in the latest edition of Remington's Pharmaceutical Sciences (Maack Publishing Co., Easton, Pa.).

The actual dosage depend on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, preferably of from about 1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

### Methods of Therapy

The diazabicyclic aryl derivatives of the present invention are valuable nicotinic and monoamine receptor modulators, and therefore useful for the treatment of a range of ailments involving cholinergic dysfunction as well as a range of disorders responsive to the action of nAChR modulators.

In another aspect the invention provides a method for the treatment, prevention or alleviation of a disease or a disorder or a condition of a living animal body, including a human, which disease, disorder or condition is responsive to modulation of cholinergic receptors and/or monoamine receptors, and which method comprises administering to such a living animal body, including a human, in need thereof an effective amount of an diazabicyclic aryl derivative of the invention.

In the context of this invention the term “treatment” covers treatment, prevention, prophylaxis or alleviation, and the term “disease” covers illnesses, diseases, disorders and conditions related to the disease in question.

The preferred indications contemplated according to the invention are those stated above.

It is at present contemplated that suitable dosage ranges are 0.1 to 1000 milligrams daily, 10-500 milligrams daily, and especially 30-100 milligrams daily, dependent as usual upon the exact mode of administration, form in which administered, the indication toward which the administration is directed, the subject involved and the body weight of the subject involved, and further the preference and experience of the physician or veterinarian in charge.

A satisfactory result can, in certain instances, be obtained at a dosage as low as 0.005 mg/kg i.v. and 0.01 mg/kg p.o. The upper limit of the dosage range is about 10 mg/kg i.v. and 100 mg/kg p.o. Preferred ranges are from about 0.001 to about 1 mg/kg i.v. and from about 0.1 to about 10 mg/kg p.o.

### EXAMPLES

The invention is further illustrated with reference to the following examples, which are not intended to be in any way limiting to the scope of the invention as claimed.

### Example 1

### Preparatory Example

All reactions involving air sensitive reagents or intermediates were performed under nitrogen and in anhydrous solvents.

### 1,4-Diazabicyclo[3.2.2]nonan-3-one (Intermediate Compound)

32.33 g (200 mmol) of 3-Quinuclidinone hydrochloride was dissolved in 75 ml of water, and to the solution of hydroxylamine hydrochloride (16.4 g; 236 mmol) and CH₃CO₂Na.3H₂O (80 g; 588 mmol) was added. The mixture was stirred at 70° C. for 1 hour. Then NaCl (10 g) was dissolved in the mixture and was cooled to 0° C. Separated crystals were filtered and carefully dried. The thus obtained crude 3-quinuclidone oxime (approx. 30 g) was used in the next step of the synthesis without further purification.

Polyphosphoric acid (180 g) of was heated to 100° C. and crude 3-quinuclidone oxime (30 g) was added portion-wise. The reaction mixture was heated at 130° C. for 20 minutes. The mixture was cooled to room temperature, and 50 ml of water was added. The mass was carefully homogenised, the mixture was poured into of ice (100 g). The mixture was made alkaline (pH 12) by adding sodium hydroxide. The mixture was extracted with chloroform (2×400 ml). The extract was dried over sodium sulphate and the solvent was removed under reduced pressure.

Yield of the mixture of the products 1,4-diazabicyclo[3.2.2]nonan-3-one and 1,3-diazabicyclo[3.2.2]nonan-4-one was 19.02 g (68%). The mixture of isomers was crystallized from 80 ml of dioxane to yield 1,4-diazabicyclo[3.2.2]nonan-3-one (5.12 g; 18%). The solvent from filtrate was distilled off, flash chromatography (with acetone) of the residue gave of 1,3-diazabicyclo[3.2.2]nonan-4-one (8.91 g; 32%).

### 1,4-Diazabicyclo[3.2.2]nonane [J. Med. Chem. 1993 36 2311-2320](Intermediate Compound)

1,4-Diazabicyclo[3.2.2]nonan-3-one (5.12 g; 36 mmol) was dissolved in tetrahydrofuran (50 ml), lithium aluminium hydride 2.28 g (60 mmol) was added to the solution and the reaction mixture was refluxed for 36 hours. After cooling the reaction mixture to room temperature, water (2.3 ml) was added dropwise and the mixture was filtered. The solvent was removed from the filtrate by rotavapor at reduced pressure. The formed substance was distilled with Kugelrohr (0.5 mBar; 70° C.). Yield of the title compound 3.11 g (68%).

### Method A

### 1-{4-[5-(1,4-Diaza-bicyclo[3.2.2]nonane-4-carbonyl)-furan-2-yl]-phenyl}-3-(2-aminophenyl)-urea Free Base (Compound A1)

A mixture of 1-{4-[5-(1,4-diaza-bicyclo[3.2.2]nonane-4-carbonyl)-furan-2-yl]-phenyl}-3-(2-nitrophenyl)-urea (0.63 g; 1.32 mmol), palladium on carbon (0.60 g; 5%) and ethanol (50 ml) was stirred under hydrogen. The crude mixture was filtered and purified by silica gel chromatography, using a mixture of dichloromethane:methanol (9:1) and 1% methanol as eluent. Yield 0.50 g (85%). Mp. 174° C.

### (1,4-Diaza-bicyclo[3.2.2]non-4-yl)-5-(4-aminophenyl)-furan-2-yl-methanone Fumaric Acid Salt (Intermediate Compound)

The title compound was prepared according to method A from (1,4-diaza-bicyclo[3.2.2]non-4-yl)-5-(4-nitrophenyl)-furan-2-yl-methanone. Mp. 227.8° C.

### 4-Amino-N-{4-[5-(1,4-diaza-bicyclo[3.2.2]nonane-4-carbonyl)-furan-2-yl]-phenyl}-benzamide Free Base (Compound A2)

The title compound was prepared according to Method A from N-{4-[5-(1,4-Diaza-bicyclo[3.2.2]nonane-4-carbonyl)-furan-2-yl]-phenyl}-4-nitro-benzamide. Mp. 151° C.

### 3-Amino-N-{4-[5-(1,4-diaza-bicyclo[3.2.2]nonane-4-carbonyl)-furan-2-yl]-phenyl}-benzamide Free Base (Compound A3)

The title compound was prepared according to Method A from N-{4-[5-(1,4-Diaza-bicyclo[3.2.2]nonane-4-carbonyl)-furan-2-yl]-phenyl}-3-nitro-benzamide. Mp. 249-252° C.

### (1,4-Diaza-bicyclo[3.2.2]non-4-yl)-5-(4-nitrophenyl)-furan-2-yl-methanone hydrochloric Acid Salt (Intermediate Compound)

A mixture of 5-(4-nitrophenyl)-2-furoyl chloride (1.0 g; 4.0 mmol), 1,4-diazabicyclo[3.2.2]nonane (0.50 g; 4.0 mmol) and 1,2-dimethoxyethane (20 ml) was stirred for 15 hours at room temperature. The title compound was filtered. Yield 1.4 g (93%). Mp. 298.2° C.

### 5-(4-Nitrophenyl)-2-furoyl chloride (Intermediate Compound)

Was prepared by stirring a mixture of 5-(4-nitrophenyl)-2-furoic acid (1.0 g; 4.3 mmol) and thionyl chloride (10 ml) at reflux for 2 hours. The mixture was evaporated and co-evaporated with anhydrous toluene. The acid chloride was used without further purification.

### Method B

### 5-Hydroxy-1-{4-[5-(1-oxy-1,4-diaza-bicyclo[3.2.2]nonane-4-carbonyl)-furan-2-yl]-phenyl}-1,5-dihydro-pyrrol-2-one (N-oxide) (Compound B1)

A mixture of (1,4-diaza-bicyclo[3.2.2]non-4-yl)-5-(4-aminophenyl)-furan-2-yl-methasone (0.50 g; 1.6 mmol), maleic anhydride (0.24 g; 2.4 mmol) and dichloromethane (10 ml) was stirred for 4 hours at room temperature. The mixture was filtered and the title compound was isolated. Yield 0.48 g (73%). Mp. 191° C.

### 1-{4-[5-(1,4-Diaza-bicyclo[3.2.2]nonane-4-carbonyl)-furan-2-yl]-phenyl}-3-(2-acetylaminophenyl)-urea Fumaric Acid Salt (Compound B2)

A mixture of 1-{4-[5-(1,4-Diaza-bicyclo[3.2.2]nonane-4-carbonyl)-furan-2-yl]-phenyl}-3-(2-aminophenyl)-urea (0.21 g; 0.47 mmol), acetic anhydride (144 mg; 1.42 mmol) and dichloromethane (20 ml) was stirred for 4 hours. Aqueous sodium hydroxide (10 ml; 1M) was added followed by extraction with dichloromethane (3×10 ml). The crude mixture was purified by silica gel chromatography, using a mixture of dichloromethane:methanol (9:1) and 1% methanol as eluent. Yield: 174 mg (79%). The corresponding salt was obtained by addition of a diethyl ether and methanol mixture (9:1) saturated with fumaric acid Mp. 159-169° C.

### Method C

### 1-{4-[5-(1,4-Diaza-bicyclo[3.2.2]nonane-4-carbonyl)-furan-2-yl]-phenyl}-3-phenyl-urea Free Base (Compound C1)

A mixture of (1,4-diaza-bicyclo[3.2.2]non-4-yl)-5-(4-aminophenyl)-furan-2-yl-methanone (0.50 g; 1.6 mmol), phenyl-isocyanate (498 mg; 4.18 mmol) and dichloromethane (50 ml) was stirred for 15 hours. Aqueous sodium hydroxide (10 ml; 1M) was added followed by extraction with dichloromethane (3×10 ml). The crude mixture was purified by silica gel chromatography, using a mixture of dichloromethane:methanol (9:1) and 1% methanol as eluent. Yield 0.16 g (23%). Mp. 153-164° C.

### 1-{4-[5-(1,4-Diaza-bicyclo[3.2.2]nonane-4-carbonyl)-furan-2-yl]-phenyl}-3-(2-nitrophenyl)-urea Fumaric Acid Salt (Compound C2)

The title compound was prepared according to Method C from 2-nitrophenylisocyanate. Mp. 198-202° C.

### 1-{4-[5-(1,4-Diaza-bicyclo[3.2.2]nonane-4-carbonyl)-furan-2-yl]-phenyl}-3-ethyl-urea Fumaric Acid Salt (Compound C3)

The title compound was prepared according to Method C from ethylisocyanate. Mp. 167-171° C.

### 1-{4-[5-(1,4-Diaza-bicyclo[3.2.2]nonane-4-carbonyl)-furan-2-yl]-phenyl}-3-phenylthiourea Free Base (Compound C4)

The title compound was prepared according to Method C from phenylisothiocyanate. Mp. 171.4-174.7° C.

### 1-{4-[5-(1,4-Diaza-bicyclo[3.2.2]nonane-4-carbonyl)-furan-2-yl]-phenyl}-3-(5-chloro-2-methoxy phenyl-urea Free Base (Compound C5)

The title compound was prepared according to Method C from 5-chloro-2-methoxyphenylisocyanate. Isolated as an oil.

### 1-{4-[5-(1,4-Diaza-bicyclo[3.2.2]nonane-4-carbonyl)-furan-2-yl]-phenyl}-3-benzyl-urea Free Base (Compound C6)

The title compound was prepared according to Method C from benzylisocyanate. Isolated as an oil.

### 1-{4-[5-(1,4-Diaza-bicyclo[3.2.2]nonane-4-carbonyl)-furan-2-yl]-phenyl}-1′-benzylaminocarbonyl-3-benzyl-urea Free Base (Compound C7)

The title compound was prepared according to Method C from benzylisothiocyanate. Mp. 105-130° C.

### 1-{4-[5-(1,4-Diaza-bicyclo[3.2.2]nonane-4-carbonyl)-furan-2-yl]-phenyl}-3-(2-chlorophenyl)-urea Free Base (Compound C8)

The title compound was prepared according to Method C from 2-chlorophenylisocyanate. Mp. 200-211° C. (decomp.).

### 1-{4-[5-(1,4-Diaza-bicyclo[3.2.2]nonane-4-carbonyl)-furan-2-yl]-phenyl}-3-phenyl-urea Free Base (Compound C9)

The title compound was prepared according to Method C from (1,4-diaza-bicyclo[3.2.2]non-4-yl)-5-(3-aminophenyl)-furan-2-yl-methanone and phenylisocyanate. Mp. 125-130° C.

### 1-{4-[5-(1,4-Diaza-bicyclo[3.2.2]nonane-4-carbonyl)-furan-2-yl]-phenyl}-3-(2-fluorophenyl)-urea Free Base (Compound C10)

The title compound was prepared according to Method C from 2-fluorophenylisocyanate. Mp. 241° C. (decomp.).

### 1-{4-[5-(1,4-Diaza-bicyclo[3.2.2]nonane-4-carbonyl)-furan-2-yl]-phenyl}-3-(3-fluorophenyl)-urea Free Base (Compound C11)

The title compound was prepared according to Method C from 3-fluorophenylisocyanate. Mp. 230° C.

### 1-{4-[5-(1,4-Diaza-bicyclo[3.2.2]nonane-4-carbonyl)-furan-2-yl]-phenyl}-3-(2-trifluoromethylphenyl)-urea Free base (Compound C12)

The title compound was prepared according to Method C from 2-trifluoromethylphenylisocyanate. Mp. 253° C. (decomp.).

### 1-[2-(3-{4-[5-(1,4-Diaza-bicyclo[3.2.2]nonane-4-carbonyl)-furan-2-yl]-phenyl}-ureido)-phenyl]-3-ethyl-urea Fumaric Acid Salt (Compound C13)

The title compound was prepared according to Method C from 1-(2-amino-phenyl)-3-{4-[5-(1,4-diaza-bicyclo[3.2.2]nonane-4-carbonyl)-furan-2-yl]-phenyl}-urea and ethylisocyanate. Mp. 162.5-165.5° C.

### 1-{4-[5-(1,4-Diaza-bicyclo[3.2.2]nonane-4-carbonyl)-furan-2-yl]-phenyl}-3-(3-trifluoromethylphenyl)-urea Free Base (Compound C14)

The title compound was prepared according to Method C from 3-trifluoromethylphenylisocyanate. Mp. 171° C.

### 1-{3-[5-(1,4-Diaza-bicyclo[3.2.2]nonane-4-carbonyl)-furan-2-yl]-phenyl}-3-ethyl-urea Fumaric Acid Salt (Compound C15)

The title compound was prepared according to Method C from ethylisocyanate. Mp. 158-161° C.

### N-{4-[5-(1,4-Diaza-bicyclo[3.2.2]nonane-4-carbonyl)-furan-2-yl]-phenyl}-isonicotinamide Fumaric Acid Salt (Compound C16)

The title compound was prepared according to Method C from [5-(4-amino-phenyl)-furan-2-yl]-(1,4-diaza-bicyclo[3.2.2]non-4-yl)-methanone. Mp. 250-253.5° C.

### Method D

### N-{4-[5-(1,4-Diaza-bicyclo[3.2.2]nonane-4-carbonyl)-furan-2-yl]-phenyl}-benzamide Free Base (Compound D1)

To a mixture of (1,4-diaza-bicyclo[3.2.2]non-4-yl)-5-(4-aminophenyl)-furan-2-yl-methanone (0.50 g, 1.6 mmol), diisopropylamine (415 mg, 3.2 mmol) and dichloromethane (100 ml), was added at 0° C.: benzoyl chloride 0.586 g, 4.18 mmol) and stirred for 15 h at room temperature. Aqueous sodium hydroxide (10 ml; 1M) was added followed by extraction with dichloromethane (3×10 ml). The crude mixture was purified by silica gel chromatography, using a mixture of dichloromethane:methanol (9:1) and 1% methanol as eluent. Yield 0.50 g (75%). Mp. 254° C.

### N-{3-[5-(1,4-Diaza-bicyclo[3.2.2]nonane-4-carbonyl)-furan-2-yl]-phenyl}-benzamide Fumaric Acid Salt (Compound D2)

The title compound was prepared according to Method D from (1,4-diaza-bicyclo[3.2.2]non-4-yl)-5-(3-aminophenyl)-furan-2-yl-methanone. Mp. 201-204° C.

### N-{4-[5-(1,4-Diaza-bicyclo[3.2.2]nonane-4-carbonyl)-furan-2-yl]-phenyl}-2-nitro-benzamide hydrochloric acid salt (Compound D3)

The title compound was prepared according to Method D from (1,4-diaza-bicyclo[3.2.2]non-4-yl)-5-(4-aminophenyl)-furan-2-yl-methanone. Mp. 283° C.

### N-{4-[5-(1,4-Diaza-bicyclo[3.2.2]nonane-4-carbonyl)-furan-2-yl]-phenyl}-4-nitro-benzamide hydrochloric acid salt (Compound D4)

The title compound was prepared according to Method D from (1,4-diaza-bicyclo[3.2.2]non-4-yl)-5-(4-aminophenyl)-furan-2-yl-methanone. Mp. 195-210° C.

### N-{4-[5-(1,4-Diaza-bicyclo[3.2.2]nonane-4-carbonyl)-furan-2-yl]-phenyl}-3-nitro-benzamide hydrochloric acid salt (Compound D5)

The title compound was prepared according to Method D from (1,4-diaza-bicyclo[3.2.2]non-4-yl)-5-(4-aminophenyl)-furan-2-yl-methanone. Mp.>300° C.

### Method E

### (1,4-Diaza-bicyclo[3.2.2]non-4-yl)-[5-(4-pyrrol-1-yl-phenyl)-furan-2-yl]-methanone Fumaric Acid Salt (Compound E1)

A mixture of 1,4-diaza-bicyclo[3.2.2]non-4-yl)-5-(4-aminophenyl)-furan-2-yl-methanone (0.50 g, 1.6 mmol), 2,5-dimethoxytetrahydrofuran (4.24 g, 64 mmol), acetic acid (0.5 ml) and dioxane (30 ml) was stirred at reflux for 20 h. Aqueous sodium hydroxide (10 ml; 1M) was added followed by extraction with dichloromethane (3×10 ml). The crude mixture was purified by silica gel chromatography, using a mixture of dichloromethane:methanol (9:1) and 1% methanol as eluent. The corresponding salt was obtained by addition of a diethyl ether and methanol mixture (9:1) saturated with fumaric acid. Yield 0.33 g (43%). Mp. 222° C.

### Method F

### (1,4-Diaza-bicyclo[3.2.2]non-4-yl)-{5-[4-(3-nitro-pyridin-2-ylamino)-phenyl]-furan-2-yl}-methanone Fumaric Acid Salt (Compound F1)

A mixture of 1,4-diaza-bicyclo[3.2.2]non-4-yl)-5-(4-aminophenyl)-furan-2-yl-methanone (0.50 g, 1.6 mmol), 2-chloro-3-nitropyridine (0.25 g, 1.6 mmol), cesium carbonate (0.78 g, 2.41 mmol) and NMP (0.5 ml) was stirred at 80° C. for 3 days. Aqueous sodium hydroxide (10 ml; 1M) was added followed by extraction with dichloromethane (3×10 ml). The crude mixture was purified by silica gel chromatography, using a mixture of dichloromethane:methanol (9:1) and 1% methanol as eluent. Yield 110 mg (16%). The corresponding salt was obtained by addition of a diethyl ether and methanol mixture (9:1) saturated with fumaric acid. Mp. 223° C.

### Example 2

### In vitro Inhibition of ³H-α-Bungarotoxine Binding in Rat Brain

In this example the affinity of the compounds of the invention for binding to α₇-subtype of nicotinic receptors is determined.

α-Bungarotoxine is a peptide isolated from the venom of the Elapidae snake Bungarus multicinctus. It has high affinity for neuronal and neuromuscular nicotinic receptors, where it acts as a potent antagonist. ³H-α-Bungarotoxine labels nicotinic acetylcholine receptors formed by the α₇ subunit isoform found in brain and the α₁ isoform in the neuromuscular junction.

### Tissue Preparation

Preparations are performed at 0-4° C. Cerebral cortices from male Wistar rats (150-250 g) are homogenised for 10 seconds in 15 ml of 20 mM Hepes buffer containing 118 mM NaCl, 4.8 mM KCl, 1.2 mM MgSO₄ and 2.5 mM CaCl₂ (pH 7.5) using an Ultra-Turrax homogeniser. The tissue suspension is subjected to centrifugation at 27,000×g for 10 minutes. The supernatant is discarded and the pellet is washed twice by centrifugation at 27,000×g for 10 minutes in 20 ml of fresh buffer, and the final pellet is then re-suspended in fresh buffer containing 0.01% BSA (35 ml per g of original tissue) and used for binding assays.

### Assay

Aliquots of 500 μl of homogenate are added to 25 μl of test solution and 25 μl of ³H-α-bungarotoxine (2 nM, final concentration) and mixed and incubated for 2 hours at 37° C. Non-specific binding is determined using (−)-nicotine (1 mM, final concentration). After incubation, the samples are added 5 ml of ice-cold Hepes buffer containing 0.05% PEI and poured directly onto Whatman GF/C glass fibre filters (pre-soaked in 0.1% PEI for at least 6 hours) under suction, and immediately washed with 2×5 ml ice-cold buffer.

The amount of radioactivity on the filters is determined by conventional liquid scintillation counting. Specific binding is total binding minus non-specific binding.

The test value is given as an IC₅₀ (the concentration of the test substance which inhibits the specific binding of ³H-α-bungarotoxin by 50%).

The results of these experiments are presented in Table 1 below.

TABLE 1
Inhibition of ³H-α-Bungarotoxine Binding

| Compound No. | IC₅₀ (μM) |
|---|---|
| A1 | 0.0012 |
| B2 | 0.0016 |
| C3 | 0.00056 |

1.-26. (canceled)

27. An diazabicyclic aryl derivative represented by Formula IV 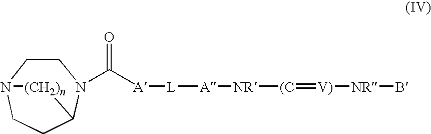
any of its enantiomers or any mixture of its enantiomers, or a prodrug, or a pharmaceutically-acceptable addition salt thereof, wherein
n is 1, 2 or 3;
A′ and A″, independently of one another, represent an aromatic monocyclic and/or polycyclic, carbocyclic and/or heterocyclic group, optionally substituted one or more times with substituents selected from the group consisting of alkyl, cycloalkyl, cycloalkyl-alkyl, hydroxy, alkoxy, hydroxyalkoxy, alkoxy-alkyl, alkoxy-alkoxy, cycloalkoxy, cycloalkoxy-alkyl, cycloalkoxy-alkoxy, halo, trihalomethyl, trihalomethoxy, cyano, nitro, amino, carboxy, amino-carbonyl (carbamoyl), sulfamoyl and phenyl; or with another monocyclic or polycyclic, carbocyclic or heterocyclic group; which additional monocyclic or polycyclic, carbocyclic or heterocyclic group may optionally be substituted one or more times with substituents selected from the group consisting of alkyl, cycloalkyl, cycloalkyl-alkyl, hydroxy, alkoxy, hydroxyalkoxy, alkoxy-alkyl, alkoxy-alkoxy, cycloalkoxy, cycloalkoxy-alkyl, cycloalkoxy-alkoxy, halo, trihalomethyl, trihalomethoxy, cyano, nitro, amino, carboxy, amino-carbonyl (carbamoyl), sulfamoyl and phenyl;
R′ represents hydrogen, alkyl or a group of formula —(C═V)—NR″—B′;
R″ represents hydrogen, alkyl, phenyl or benzyl;
V represents O, S or NR′″; wherein R′″ represents hydrogen, alkyl or cyano;
B′ represents hydrogen, alkyl, alkenyl, alkynyl, cycloalkyl, cycloalkenyl, phenyl, benzyl or a monocyclic heterocyclic group; which phenyl, benzyl and heterocyclic groups are optionally substituted one or more times with substituents selected from the group consisting of alkyl, cycloalkyl, cycloalkyl-alkyl, hydroxy, alkoxy, hydroxyalkoxy, alkoxy-alkyl, alkoxy-alkoxy, cycloalkoxy, cycloalkoxy-alkyl, cycloalkoxy-alkoxy, halo, trihalomethyl, trihalomethoxy, cyano, nitro, amino, oxo, carboxy, amino-carbonyl (carbamoyl), N-alkyl-amino-carbonyl (alkyl-carbamoyl), N,N-dialkyl-amino-carbonyl, alkyl-carbonyl-amino, amino-carbonyl-amino (ureido), N-alkyl-amino-carbonyl-amino (N-alkyl-ureido), N,N-dialkyl-amino-carbonyl-amino (N,N-dialkyl-ureido), sulfamoyl, phenyl and benzyl; and
L represents a single (covalent) bond (i.e. L is absent), or a linking group selected from —CH₂—, —CH₂—CH₂—, —CH═CH—, —C≡C—, —Y—(CH₂)ₘ—, —(CH₂)ₘ—Y—, —CONR″″—, —NR″″CO—, —NR″″SO₂— and —SO₂NR″″—, wherein Y represents —O—, —S—, —SCH₂—, —SO—, —SO₂—, —NR″″—, R″″ represents hydrogen or alkyl; and m is 0, 1, 2 or 3.

28. The diazabicyclic aryl derivative of claim 27, wherein
L represents a single (covalent) bond (i.e. L is absent);
R′ represents hydrogen, alkyl or a group of formula —(C═V)—NR″—B′;
R″ represents hydrogen, alkyl, phenyl or benzyl;
V represents O, S or NR′″; wherein R′″ represents hydrogen, alkyl or cyano; and
B′ represents hydrogen, alkyl, alkenyl, alkynyl, cycloalkyl, cycloalkenyl, phenyl, benzyl or a monocyclic heterocyclic group; which phenyl, benzyl and heterocyclic groups are optionally substituted one, two or three times with substituents selected from the group consisting of alkyl, cycloalkyl, cycloalkyl-alkyl, hydroxy, alkoxy, hydroxyalkoxy, alkoxy-alkyl, alkoxy-alkoxy, cycloalkoxy, cycloalkoxy-alkyl, cycloalkoxy-alkoxy, halo, trihalomethyl, trihalomethoxy, cyano, nitro, amino, oxo, carboxy, amino-carbonyl (carbamoyl), N-alkyl-amino-carbonyl (alkyl-carbamoyl), N,N-dialkyl-amino-carbonyl, alkyl-carbonyl-amino, amino-carbonyl-amino (ureido), N-alkyl-amino-carbonyl-amino (N-alkyl-ureido), N,N-dialkyl-amino-carbonyl-amino (N,N-dialkyl-ureido), sulfamoyl, phenyl and benzyl.

29. The diazabicyclic aryl derivative of claim 28, wherein B′ represents alkyl, phenyl or benzyl; which phenyl and benzyl groups are optionally substituted one or two times with hydroxy, alkoxy, halo, trifluoromethyl, nitro, amino, alkyl-carbonyl-amino, amino-carbonyl-amino (ureido), N-alkyl-amino-carbonyl-amino (N-alkyl-ureido) and/or N,N-dialkyl-amino-carbonyl-amino (N,N-dialkyl-ureido).

30. The diazabicyclic aryl derivative of claim 27, wherein
n is 2;
L represents a single (covalent) bond (i.e. L is absent);
A′ represents a furanyl, oxazolyl, oxadiazolyl, thiazolyl or pyridazinyl group;
A″ represents a phenyl group; and
R′ represents hydrogen, alkyl or —(C═O)—NH—B′—;
R″ represents hydrogen, alkyl, phenyl or benzyl;
V represents O, S or NH; and
B′ represents a group of formula —CH₃, —CH₂CH₃, —CH═CH₂, —CH═CH—CH═CH₂, cyclopenta-1-enyl cyclopenta-2,4-dienyl, phenyl or benzyl; which phenyl and benzyl may optionally be substituted one or two times with alkyl, hydroxy, alkoxy, halo, trihalomethyl, trihalomethoxy, cyano, nitro, amino, amino-carbonyl (amido), N-alkyl-amino-carbonyl (N-alkyl-amido), N,N-dialkyl-amino-carbonyl (N,N-dialkyl-amido) and/or alkyl-carbonyl-amino.

31. The diazabicyclic aryl derivative of claim 27, wherein
n is 2;
L represents a single (covalent) bond (i.e. L is absent);
A′ represents a furanyl, oxazolyl, oxadiazolyl, thiazolyl or pyridazinyl group;
A″ represents a phenyl group; and
R′ represents hydrogen, alkyl or —(C═O)—NH—B′—;
R″ represents hydrogen, alkyl, phenyl or benzyl;
V represents O, S or NH; and
B′ represents a group of formula —CH₃, —CH₂CH₃, —CH═CH₂, —CH═CH—CH═CH₂, cyclopenta-1-enyl cyclopenta-2,4-dienyl, phenyl or benzyl; which phenyl and benzyl may optionally be substituted one or two times with alkyl, hydroxy, alkoxy, halo, trihalomethyl, trihalomethoxy, cyano, nitro, amino, amino-carbonyl (amido), N-alkyl-amino-carbonyl (N-alkyl-amido), N,N-dialkyl-amino-carbonyl (N,N-dialkyl-amido) and/or alkyl-carbonyl-amino.

32. The diazabicyclic aryl derivative of claim 31, wherein
B′ represents alkyl, phenyl, benzyl or pyridyl; which phenyl, benzyl and pyridyl groups are optionally substituted one or two times with substituents selected from the group consisting of hydroxy, alkoxy, halo, trifluoromethyl, nitro, amino, alkyl-carbonyl-amino, N-alkyl-amino-carbonyl-amino (N-alkyl-ureido), N,N-dialkyl-amino-carbonyl-amino (N,N-dialkyl-ureido) and sulfamoyl.

33. The diazabicyclic aryl derivative of claim 32, which is
1-{4-[5-(1,4-Diaza-bicyclo[3.2.2]nonane-4-carbonyl)-furan-2-yl]-phenyl}-3-ethyl-urea;
1-{4-[5-(1,4-Diaza-bicyclo[3.2.2]nonane-4-carbonyl)-furan-2-yl]-phenyl}-3-phenyl-urea;
1-{4-[5-(1,4-Diaza-bicyclo[3.2.2]nonane-4-carbonyl)-furan-2-yl]-phenyl}-3-(2-nitrophenyl)-urea;
1-{4-[5-(1,4-Diaza-bicyclo[3.2.2]nonane-4-carbonyl)-furan-2-yl]-phenyl}-3-(2-acetylaminophenyl)-urea;
1-{4-[5-(1,4-Diaza-bicyclo[3.2.2]nonane-4-carbonyl)-furan-2-yl]-phenyl}-3-(2-aminophenyl)-urea;
1-{4-[5-(1,4-Diaza-bicyclo[3.2.2]nonane-4-carbonyl)-furan-2-yl]-phenyl}-3-(5-chloro-2-methoxyphenyl)-thiourea;
1-{4-[5-(1,4-Diaza-bicyclo[3.2.2]nonane-4-carbonyl)-furan-2-yl]-phenyl}-3-(5-chloro-2-methoxy-phenyl)-urea;
1-{4-[5-(1,4-Diaza-bicyclo[3.2.2]nonane-4-carbonyl)-furan-2-yl]-phenyl}-3-benzyl-urea;
1-{4-[5-(1,4-Diaza-bicyclo[3.2.2]nonane-4-carbonyl)-furan-2-yl]-phenyl}-1′-benzylaminocarbonyl-3-benzyl-urea;
1-{4-[5-(1,4-Diaza-bicyclo[3.2.2]nonane-4-carbonyl)-furan-2-yl]-phenyl}-1′-benzylaminocarbonyl-3-benzyl-urea;
1-{4-[5-(1,4-Diaza-bicyclo[3.2.2]nonane-4-carbonyl)-furan-2-yl]-phenyl}-3-(2-chlorophenyl)-urea;
1-{3-[5-(1,4-Diaza-bicyclo[3.2.2]nonane-4-carbonyl)-furan-2-yl]-phenyl}-3-phenyl-urea;
1-{4-[5-(1,4-Diaza-bicyclo[3.2.2]nonane-4-carbonyl)-furan-2-yl]-phenyl}-3-(2-fluorophenyl)-urea;
1-{4-[5-(1,4-Diaza-bicyclo[3.2.2]nonane-4-carbonyl)-furan-2-yl]-phenyl}-3-(3-fluorophenyl)-urea;
1-{4-[5-(1,4-Diaza-bicyclo[3.2.2]nonane-4-carbonyl)-furan-2-yl]-phenyl}-3-(2-trifluoromethylphenyl)-urea;
1-[2-(3-{4-[5-(1,4-Diaza-bicyclo[3.2.2]nonane-4-carbonyl)-furan-2-yl]-phenyl}-ureido)-phenyl]-3-ethyl-urea;
1-{4-[5-(1,4-Diaza-bicyclo[3.2.2]nonane-4-carbonyl)-furan-2-yl]-phenyl}-3-(3-trifluoromethylphenyl)-urea; or
1-{3-[5-(1,4-Diaza-bicyclo[3.2.2]nonane-4-carbonyl)-furan-2-yl]-phenyl}-3-ethyl-urea;
or an enantiomer or a mixture of its enantiomers, or a pharmaceutically-acceptable addition salt thereof.

34. A pharmaceutical composition comprising a therapeutically effective amount of a diazabicyclic aryl derivative of claim 27, or a pharmaceutically-acceptable addition salt thereof, together with at least one pharmaceutically-acceptable carrier or diluent.

35. A method of treatment, prevention or alleviation of a disease or a disorder or a condition of a living animal body, including a human, which disorder, disease or condition is responsive to modulation of cholinergic receptors, which method comprises the step of administering to such a living animal body in need thereof a therapeutically effective amount of a diazabicyclic aryl derivative of any one of claim 27.

36. The method according to claim 35, wherein the disease, disorder or condition relates to the central nervous system.

37. The method according to claim 36, wherein the disease, disorder or condition is anxiety, cognitive disorders, learning deficit, memory deficits and dysfunction, Alzheimer's disease, attention deficit, attention deficit hyperactivity disorder, Parkinson's disease, Huntington's disease, Amyotrophic Lateral Sclerosis, Gilles de la Tourette's syndrome, depression, mania, manic depression, schizophrenia, obsessive compulsive disorders (OCD), panic disorders, eating disorders such as anorexia nervosa, bulimia and obesity, narcolepsy, nociception, AIDS-dementia, senile dementia, periferic neuropathy, autism, dyslexia, tardive dyskinesia, hyperkinesia, epilepsy, bulimia, post-traumatic syndrome, social phobia, sleeping disorders, pseudodementia, Ganser's syndrome, pre-menstrual syndrome, late luteal phase syndrome, chronic fatigue syndrome, mutism, trichotillomania and jet-lag.

38. The method according to claim 35, wherein the disease, disorder or condition are associated with smooth muscle contractions, including convulsive disorders, angina pectoris, premature labour, convulsions, diarrhoea, asthma, epilepsy, tardive dyskinesia, hyperkinesia, premature ejaculation and erectile difficulty.

39. The method according to claim 35, wherein the disease, disorder or condition is related to the endocrine system, such as thyrotoxicosis, pheochromocytoma, hypertension and arrhythmias.

40. The method according to claim 35, wherein the disease, disorder or condition is a neurodegenerative disorders, including transient anoxia and induced neuro-degeneration.

41. The method according to claim 35, wherein the disease, disorder or condition is an inflammatory disorder, including inflammatory skin disorders such as acne and rosacea, Chron's disease, inflammatory bowel disease, ulcerative colitis and diarrhoea.

42. The method according to claim 35, wherein the disease, disorder or condition is mild, moderate or even severe pain of acute, chronic or recurrent character, as well as neuropathic pain and pain caused by migraine, postoperative pain, phantom limb pain, neuropathic pain, chronic headache, central pain, pain related to diabetic neuropathy, to post therapeutic neuralgia, or to peripheral nerve injury.

43. The method according to claim 35, wherein the disease, disorder or condition is associated withdrawal symptoms caused by termination of use of addictive substances, including nicotine containing products such as tobacco, opioids such as heroin, cocaine and morphine, benzodiazepines and benzodiazepine-like drugs and alcohol.

44. (canceled)
